(19) United States
(12) Patent Application Publication
(10) Pub. No.: US 2018/0221191 A1
(43) Pub. Date: Aug. 9, 2018

(54) HEAD POSITIONING AIDS WITH ATTACHMENTS FOR MEDICAL DEVICES

(71) Applicant: Tortle Products LLC, Greenwood Village, CO (US)

(72) Inventors: Jane SCOTT, Castle Rock, CO (US); Cameron FITCH, Franktown, CO (US); Brendan FITCH, Castle Rock, CO (US)

(21) Appl. No.: 15/753,628

(22) PCT Filed: Aug. 19, 2016
(86) PCT No.: PCT/US2016/047734

§ 371 Date: Feb. 20, 2018

Related U.S. Application Data
(60) Provisional application No. 62/207,309, filed on Aug. 19, 2015.
(60) Provisional application No. 62/221,560, filed on Sep. 21, 2015.
(60) Provisional application No. 62/321,324, filed on Apr. 12, 2016.

(51) Int. Cl. A61F 5/37; A61M 16/06

(52) U.S. Cl. CPC A61F 5/3707; A61M 16/0672; A61M 16/0694; A61M 2209/088; A61M 2240/00; A61M 2205/3606; A61M 16/0683

(57) ABSTRACT

A head positioning aid (100) includes positioning members (102) secured to a headwear member (110). The positioning members maintain an infant's head in midline. At least one strap (120) on the headwear member secures the head positioning aid to an infant's head. Optional securing devices (200) secure and orient tubing (172, 174) for CPAP and nasal cannulas to the headwear member.

### CROSS REFERENCE TO RELATED APPLICATIONS

The present application claims priority to U.S. Provisional Patent Application No. 62/321,324 filed 12 Apr. 2016 entitled “Head positioning aids with attachments for medical devices,” U.S. Provisional Patent Application No. 62/221,560 filed 21 Sep. 2015 entitled “Head positioning aids with attachments for medical devices,” and U.S. Provisional Patent Application No. 62/207,309 filed 19 Aug. 2015 entitled “Head positioning aids with attachments for medical devices,” each of which is hereby incorporated herein by reference in its entirety.

The present application is related to U.S. patent application Ser. No. 14/312,594 filed 23 Jun. 2014 entitled “Head positioning aid for premature infants,” which is hereby incorporated herein by reference in its entirety.

### TECHNICAL FIELD

The technology described herein relates to head positioning aids and methods of using the same.

### BACKGROUND

Prematurely born infants often suffer from, or are at risk of developing, numerous medical complications. For example, prematurely born infants are often at risk for intraventricular hemorrhage (IVH), which is bleeding into the fluid-filled ventricles of the brain due to fragile or immature cranial blood vessels. An estimated forty percent of prematurely born infants suffer from IVH. IVH can cause increased cranial and/or blood pressure, as well as disrupted flow of cerebrospinal fluid and hydrocephalus. IVH can also be fatal; it is the second leading cause of premature infant mortality. Long-term effects of IVH may include cognitive and developmental delays, cerebral palsy, and scoliosis.

Prematurely born infants are also often at risk for compromised cerebral blood flow and respiratory distress, and are often unable to maintain their body temperatures without assistance.

Prematurely born infants often require medical intervention to manage the aforementioned risks and to promote their health and viability. Common interventions include spinal taps, head shunts, nasal cannulas, nasal continuous positive airway pressure (CPAP), ventilator support, feeding tubes, and intravenous (IV) fluid support (such as via scalp IVs).

The standard of care includes midline supine positioning with a 30° incline for an initial 72-96 hours, which helps to reduce the risk of intraventricular hemorrhaging, as well as to promote optimal cerebral blood flow and uniform distribution of respiratory support to the lungs. This care is usually provided by rolling up soft materials, such as towels, and placing them around the infant's head to prevent it from moving. These materials are not secured to either the infant or to the surface on which the infant is placed (such as an incubator mattress), so they are easily disrupted and must be regularly repositioned to help maintain the infant in midline. These materials usually must be removed during examination of the infant, such as when taking X-rays, and later replaced. When a prematurely born infant is physiologically more stable, such as after the initial 72 to 96-hour midline supine positioning period, the infant may benefit from being positioned on the right and left side of the body. The head is maintained in midline (neutral), which helps to promote optimal cerebral blood flow.

Prematurely born infants have weak neck muscles and therefore limited ability to maintain the required positioning. They also have soft skulls and are at risk for developing dolichocephaly, a condition in which the head is disproportionately long and narrow, and which can result from external deformation related to head positioning.

Infants have delicate, sensitive skin that can easily be irritated or damaged. Medical tape placed on an infant's face or scalp, such as to secure nasal cannulas, feeding tubes, or scalp IVs, can be damaging to the skin.

The information included in this Background section of the specification, including any references cited herein and any description or discussion thereof, is included for technical reference purposes only and is not to be regarded as subject matter by which the scope of the invention as defined in the claims is to be limited.

### SUMMARY

The technology disclosed herein relates to head positioning aids. The head positioning aids may be used to assist midline positioning of prematurely born infants, treat or prevent intraventricular hemorrhage, promote optimal cerebral blood flow, promote uniform distribution of respiratory support to the lungs, and treat or prevent dolichocephaly. While the discussion herein is focused on the effectiveness of the head positioning aids when used with premature infants, it should be understood that the head positioning aids may also be used with infants of full term gestation that have medical issues that require, or would be aided by, midline positioning.

In some embodiments, the head positioning aid comprises positioning members secured to a headwear member. The headwear includes straps for securing the head positioning aid to the head of an infant. Access to the scalp for placement, removal, and monitoring of scalp IVs is maintained. The positioning members minimize movement of an infant's head, maintain an infant's head in midline, and deflect an infant's moving head back to midline. The headwear may also be constructed of a hook-receptive material to reduce the number of attached fasteners, maximize size adjustability, and maximize versatility for attaching medical devices.

In some embodiments, one or more securing devices are provided with the headwear and positioning aids. The securing devices permit attachment of medical devices such as nasal cannulas or CPAP tubes without the use of medical tape on an infant's face or elsewhere and without the use of bands around the head that cause deformation of an infant's skull.

In some embodiments, a headwear attachment strip is provided with a securing device. The strip increases the strength of the connection between some securing devices and the headwear.

In some embodiments, strap flaps are provided with the headwear and positioning aids. The strap flaps protect the skin of an infant's face from damage caused by securing devices.

In some embodiments, a support member is provided with the headwear and positioning aids. The support member supports an infant's neck and promotes an open airway.

In one implementation, a method for using the head positioning aid, such as to support an infant in a midline position, is provided. The headwear is applied to the infant's head without disturbing scalp IVs or other medical devices connected to the infant's head, which minimizes stress on the infant. Closing and fastening the straps secures the head positioning aid to the infant and reduces or prevents the unintentional movement of the headwear. When the straps are closed, a surface is provided for attachment of securing devices that secure medical devices, such as nasal cannulas and CPAP tubes, to the infant's head. The securing devices permit attachment of medical devices without the use of medical tape on an infant's face or elsewhere and without the use of bands around the head that cause deformation of an infant's skull. The positioning members are positioned on either side of the infant's head and help to prevent the head from moving. The infant's head is thereby maintained in a midline position with respect to the spine of the infant when the infant is supine.

In another implementation, a method for using the head positioning aid, such as to support an infant's head in midline and to treat or prevent dolichocephaly while the infant is lying on its side, is provided. The headwear is applied as described above. The positioning members are positioned toward the back sides of the infant's head and help to prevent the head from moving. The infant's head is thereby maintained in a midline position with respect to the spine of the infant when the infant is lying on its side. Further, the positioning members provide a rounding effect on the sides of the head (and the back of the head when in a midline position) to counter possible dolichocephaly if the head were to be merely placed on a flat surface.

In some implementations, a method for using the head positioning aid, such as to secure a medical device to the infant while maintaining the infant's head in midline, is provided. The headwear and positioning members are applied and positioned by any method described above. In one embodiment, a clamp securing device is fitted over CPAP tubes and secured to the closed hook-receptive straps of the headwear with a hook fastener. In another embodiment, a CPAP tube is seated in a slot of a cradle securing device that is secured to the closed straps. In another embodiment, a CPAP tube is seated in a slot of a bridge securing device that is secured to the closed straps. In another embodiment, a tie wrap securing device is tied around a tube, such as a nasal cannula or CPAP tube, and secured to the headwear by engaging complimentary hook-and-loop fasteners. In another embodiment, a tube is captured in a passageway through a tunnel securing device that is secured to the headwear by engaging complimentary hook-and-loop fasteners. In some embodiments, a nasal prong or mask is held in a desired location over the infant's nose by looping or tying one end of a nasal adapter strap to the prong or mask and securing the other end to the headwear.

In some methods of using the head positioning aid to secure a medical device to the infant, a headwear attachment strip is provided. The strip is positioned between a securing device and the headwear, which increases the strength of the connection between the device and the headwear.

In another implementation, a method for using the head positioning aid including a strap flap, such as to protect the skin of an infant's face, is provided. The headwear and positioning members are used according to any method described above. The strap flap is positioned between an infant's face and a securing device, which helps to protect the skin of an infant's face from damage by the device.

In another method for using the head positioning aid, the infant's head rests on the back of the headwear and the straps are not engaged. The positioning members maintain the infant's head in midline as described immediately above.

In another implementation, a method for using the head positioning aid including a support member, such as to support the neck of an infant and promote an open airway, is provided. The headwear and positioning members are used according to any method described above. The support member, which may be secured to the headwear, is positioned under the neck of an infant. The infant's neck is thereby supported in a position that promotes an open airway, which may be helpful for any prematurely born infant, including one who requires respiratory support.

In other implementations, the head positioning aid is provided as part of a kit that also includes at least one securing device. A kit allows convenient transport, storage, and laundering of the head positioning aid and other components.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention as defined in the claims is provided in the following written description of various embodiments of the invention and illustrated in the accompanying drawings.

### BRIEF DESCRIPTION OF THE DRAWINGS

FIG. 1 is a front isometric view of a head positioning aid according to one embodiment with the straps laid open.

FIG. 2 is a front isometric view of the head positioning aid of FIG. 1 with the straps folded in.

FIG. 3 is a rear elevation view of the head positioning aid of FIG. 1 with the straps laid open.

FIG. 4 is a rear elevation view of the head positioning aid of FIG. 1 with the straps folded in.

FIG. 5A is a bottom front isometric view of a securing device according to one embodiment.

FIG. 5B is a top front isometric of the securing device of FIG. 5A used in conjunction with the head positioning aid of FIG. 1 as applied to an infant's head.

FIGS. 6A and 6B are both front right isometric views of securing devices according other embodiments.

FIG. 7 is a left isometric view of a securing device of FIG. 6B used in conjunction with the head positioning aid of FIG. 1 as applied to an infant's head.

FIG. 8 is a top left isometric view of two securing devices of FIG. 6B used in conjunction with the head positioning aid of FIG. 1 as applied to an infant's head.

FIG. 9A is a top front left isometric view of a securing device according to another embodiment.

FIG. 9B is a top left isometric view of the securing device of FIG. 9A used in conjunction with the head positioning aid of FIG. 1 as applied to an infant's head.

FIG. 10 is a top front isometric view of a securing device of another embodiment in the open position.

FIG. 11 is a top front isometric view of the securing device of FIG. 10 in the closed position.

FIG. 12 is a front isometric view of the body of the securing device of FIG. 10.

FIG. 13 is a top front isometric view of the securing device of FIG. 10 in the open position used in conjunction with the head positioning aid of FIG. 1 as applied to an infant's head.

FIG. 14 is a top left isometric view of the securing device of FIG. 10 in the closed position used in conjunction with the head positioning aid of FIG. 1 as applied to an infant's head.

FIG. 15 is a front isometric view of the body of a securing device of another embodiment.

FIG. 16 is a front isometric view of the securing device of another embodiment in the open position.

FIG. 17 is a top left isometric view of the securing device of FIG. 16 used in conjunction with the head positioning aid of FIG. 1 as applied to an infant's head.

FIG. 18 is a top right isometric view of the securing device of FIG. 16 used in conjunction with the head positioning aid of FIG. 1 as applied to an infant's head.

FIG. 19 is a front isometric view of the securing device of FIG. 16 in the closed position used in conjunction with the head positioning aid of FIG. 1 as applied to an infant's head.

FIG. 20 is a top front isometric view of a nasal adapter strap according to another embodiment.

FIGS. 21 and 22 are top right isometric views of the nasal adapter strap of FIG. 20 used in conjunction with the head positioning aid of FIG. 1 as applied to an infant's head.

FIG. 23 is a top front isometric view of a chin strap according to one embodiment.

FIG. 24 is a front elevation view of a support member according to one embodiment.

### DETAILED DESCRIPTION

Head Positioning Aid

Head positioning aids primarily for use with the treatment of premature infants are disclosed herein. The head positioning aids may be generally understood as having positioning members secured to a headwear member. The positioning members maintain an infant's head in midline. In some implementations, one or more straps assist in securing the headwear member to an infant's head. In some embodiments, continuous positive airway pressure (CPAP), nasal cannula, or feeding tubes may be secured to the headwear member with the use of securing devices and without the use of medical tape on an infant's face or elsewhere. In some implementations, access to the scalp for placement, removal, or monitoring of scalp IVs and for regulating temperature is maintained while the head positioning aid is in use.

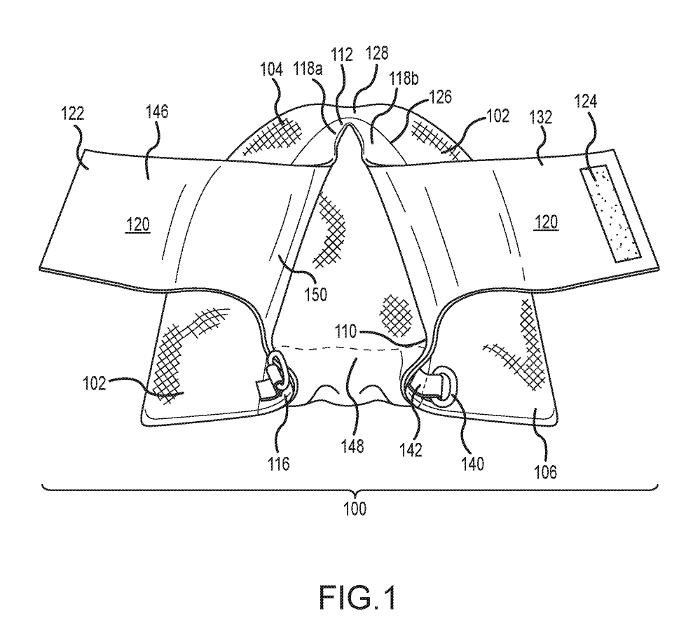

FIGS. 1-4 depict one embodiment of a head positioning aid 100. The head positioning aid 100 comprises positioning members 102 secured to a headwear member 110, which comprises at least one strap 120. While two positioning members 102 are shown in the figures as separate members spaced laterally apart from each other, a single U-shaped or V-shaped positioning member could also be used so long as two lateral support members are provided as part of the single positioning member along lateral sides of the head to prevent side-to-side movement or rolling thereof.

The positioning members 102 may be comprised of a filler. The filler may be, for example, solid foam, memory foam, stuffing, batting, down, synthetic down-like material, gel, or a combination thereof. The filler may be resilient such that it returns to its original shape, or close to its original shape, after being compressed. The filler may be of sufficient quantity, compactness, or firmness that it resists complete compression by the weight of an infant's head. The filler may be partially compressible, but has enough compression resistance that it helps each positioning member 102 minimize movement of an infant's head, maintain an infant's head in midline, and deflect an infant's moving head back to midline. The compression resistance may be matched to the size and weight of an infant's head, or to the force exerted by an infant's moving head. For example, the filler may compress to about fifty percent or less of its uncompressed thickness when weighted under an infant's head.

In some embodiments, the positioning members 102 are inflatable, such as with air. Each positioning member 102 may include a bladder connected to a valve. The valve or a tube connected to the valve is accessible from the outer surface of the positioning member 102, which allows the bladder to be inflated. The bladder may be inflated manually, such as by blowing into the valve or tube, or may be inflated mechanically, such as with a pump supplying compressed air.

Each bladder may be filled such that it has sufficient volume or firmness to resist complete compression by the weight of an infant's head. The bladders may be filled such that they help the positioning members 102 minimize movement of an infant's head, maintain an infant's head in midline, and deflect an infant's moving head back to midline. The fill amount may be matched to the size and weight of an infant's head, or to the force exerted by an infant's moving head.

In some implementations, one bladder is deflated or filled to a volume that permits the infant's head to partially or completely compress the corresponding positioning member 102 and the other bladder is inflated to a volume that directs the infant's head toward the first deflated positioning member 102. The bladders may be inflated or deflated in an opposing coordinated manner to move the head of the infant from side to side at any desired time interval.

Each positioning member 102 may include a casing, such as a fabric casing. The casing may completely or partially surround a filler or a bladder. The casing may be constructed of one or more pieces joined together by any means known in the art including, but not limited to, stitches, glue, tape, bonding, or any combination thereof. The one or more pieces may meet at one or more seams.

Each positioning member 102 may have a substantially arc or crescent shape with a truncated end. For example, each positioning member 102 may generally follow the curvature of the top, back, and/or sides of an infant's skull. The positioning members 102 may each have the same shape or have different shapes, and may each have the same size or be of different sizes. The size of the positioning members 102 may be commensurate with the size of the infant's head to which the head positioning aid is secured. Each positioning member 102 has an upper end 104 positioned at or near the apex 128 of the headwear member 110, and has a lower end 106 positioned at or near the lower portion 116 of the headwear member 110. The truncated end of the arc shape of the positioning member 102 may be at the lower end 106.

Each positioning member 102 may be the same length as, shorter than, or longer than, the length of the headwear member 110 at the location to which that positioning member 102 is secured. Thus, the upper end 104 may extend beyond the apex 128 of the headwear member 110, extend to the apex 128 of the headwear member 110, or terminate below the apex 128 of the headwear member 110. The lower end 106 may extend beyond the lower portion 116 of the headwear member 110, extend to the lower portion 116 of the headwear member 110, or terminate above the lower portion 116 of the headwear member 110.

Each positioning member 102 is elongated such that its length is greater than either its width or depth (thickness). The length of a positioning member 102 may be 2 to 6 times greater than its thickness at the lower end 106. For example, the length of a positioning member 102 may be about 4 times greater than its thickness at the lower end 106. The longitudinal elongation may help a positioning member 106 maintain an infant's head in midline and deflect an infant's moving head back to midline.

Each positioning member 102 extends outward from the outer surface of the headwear member 110. Each positioning member 102 may extend away from the headwear member 110 for the same distance along the entire length of the positioning member 102, or the extension distance (thickness) may vary along the length of the positioning member 102. Each positioning member 102 may be thicker at its lower end 106 than at its upper end 104. For example, a positioning member 102 may have nominal thickness at the upper end 104 and a functional thickness at its lower end 106. The thickness at the lower end 106 may be enough to help the positioning member 102 maintain an infant's head in midline and deflect an infant's moving head back to midline. In one embodiment, the lower end 106 is 0.5 inch to 3 inches thick.

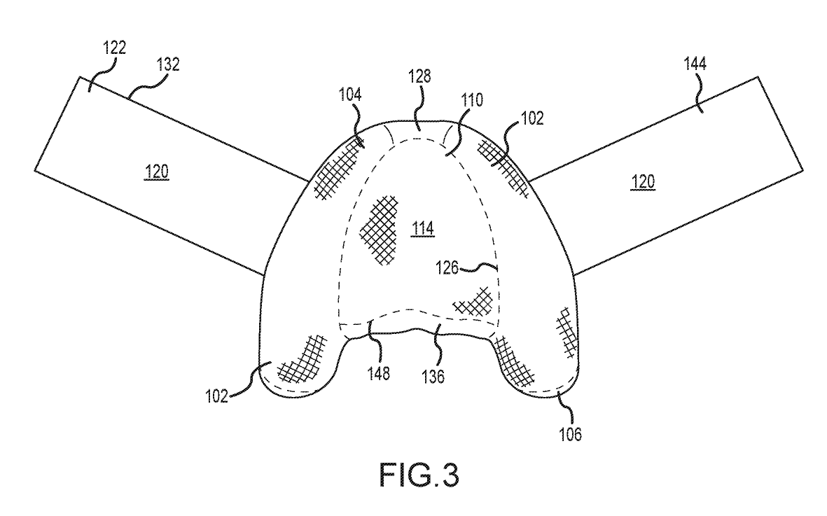

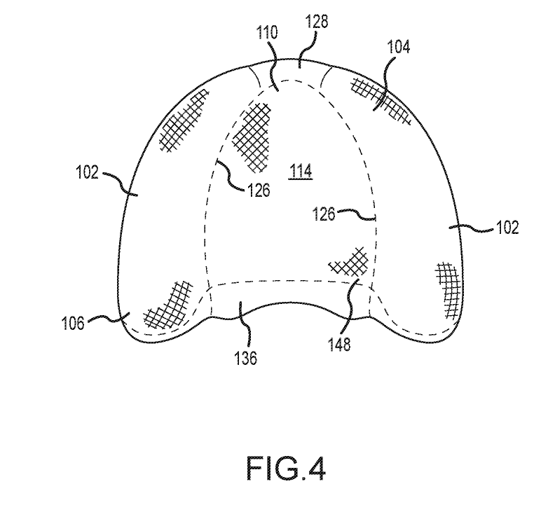

With reference to FIGS. 3 and 4, a positioning member 102 may have the same lateral distance (width or diameter) along the entire length of the positioning member 102, or the width may vary along the length of the positioning member 102. A positioning member 102 may be wider or have a larger diameter at its lower end 106 than at its upper end 104. The lower end 106 may be 1.25 to 3 times as wide as the upper end 104. For example, the lower end 106 may be about twice as wide as the upper end 104. In one embodiment, the lower end 106 is 0.25 to 2.5 inches wide.

The thickness of the lower end 106 of a positioning member 102 may be greater than, less than, or equal to, its width. In one embodiment, the thickness of the lower end 106 of a positioning member 102 is 1.25 to 3 times the width of the lower end 106 of that positioning member 102. In another embodiment, the thickness of the lower end 106 of a positioning member 102 is about twice the width of the lower end 106 of that positioning member 102.

Each positioning member 102 is secured to a headwear member 110. The overall shape of the headwear member 110 approximates at least a portion of an infant's skull. The headwear member 110 may fit closely or snugly over at least a portion of an infant's skull.

The headwear member 110 of the exemplary embodiment depicted in FIGS. 1-4 has an apex 128, a front side 112, and a back side 114. The apex 128 may include one or more closures of one or more pieces of fabric. A closure may be by any means known in the art including, but not limited to, stitches, glue, tape, bonding, fasteners, or any combination thereof. Fasteners may include, for example, buttons, snaps, hook-and-loop fasteners (e.g., Velcro®), or hook-and-eye fasteners. The closure may be formed with one or more seams.

In one embodiment, as depicted in FIGS. 3 and 4, the back side 114 includes a back lower portion 136, which may include one or more hems 148. A hem 148 may extend partially or completely around the back lower portion 136. A hem 148 may be any width. The width may be the same for the entire length of a hem 148, or the width may vary. When the head positioning aid 100 is worn by an infant, the back lower portion 136 may lie near or on the base of the infant's skull and above the nape of the neck.

In the exemplary embodiment depicted in FIGS. 1-4, the front side 112 has a lower portion 116, a lateral flap 118, and at least one strap 120. The lower portion 116 may terminate in an edge that may be smooth or rounded so as to be comfortable against an infant's head. When the head positioning aid 100 is worn by an infant, the lower portion 116 may lie near or on the infant's ears.

The lateral flap 118 extends from the apex 128 to the lower portion 116 of the headwear member 110. A front edge of the lateral flap 118 may be substantially arc-shaped with a truncated end near the lower portion 116. Thus, the width of the lateral flap 116 near the apex 128 may be narrower than the width of the same lateral flap 116 near the lower portion 116. The lateral flap 118 may include a left lateral flap 118a and a right lateral flap 118b, each of which is adjacent to one positioning member 102. When the head positioning aid 100 is worn by an infant, the lateral flap 118 may cover the side of an infant's head, such as the infant's ears.

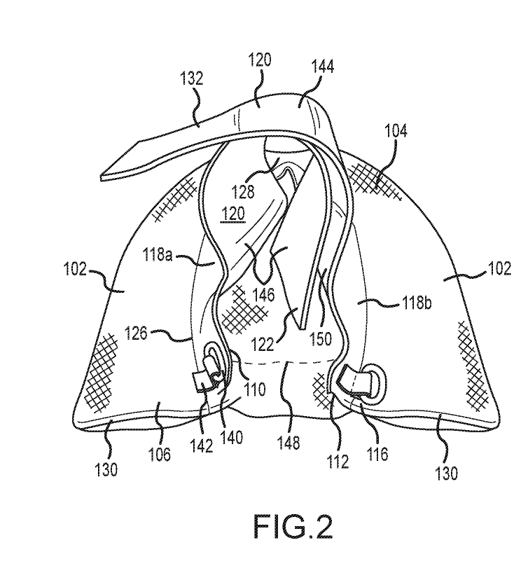

In one embodiment, at least one strap 120 is attached at a fixed end 150 to the lateral flap 118. In another embodiment, the strap 120 is an extension of the same material forming the flap 118. In the exemplary embodiment of FIGS. 1 and 2, respective straps 120 extend from each of the left lateral flap 118a and the right lateral flap 118b.

In another embodiment, the front side 112 includes at least one strap 120 and a lower portion 116. The fixed end 150 of each strap 120 is attached at or near a positioning member 102.

Each strap 120 may have a top surface 144 and a bottom surface 146. Each strap 120 may be substantially rectangular in shape with its length greater than its width. In one embodiment, each strap 120 is 1 to 8 inches in length. In one embodiment, each strap 120 is 0.5 inch to 4 inches in width.

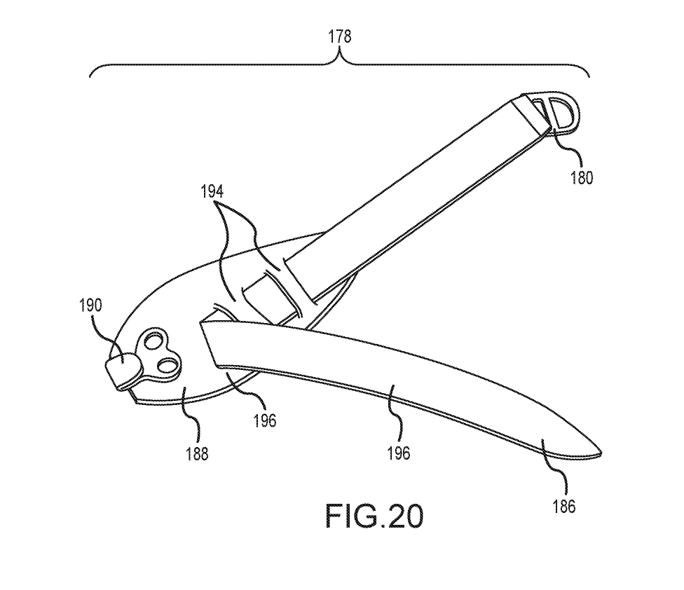

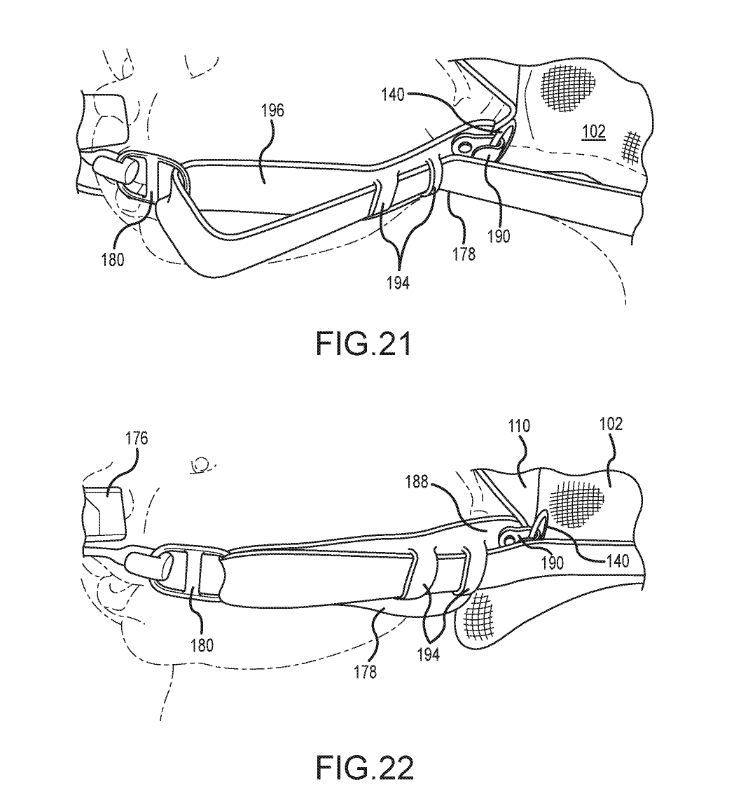

The headwear member 110 may also include a pair of retention loops 140 on the lower portion 116 of the front side 112 on or near the lateral flap 118 as depicted in FIGS. 1 and 2. The retention loops 140 may be constructed of a stiff material, such as plastic, or a soft material, such as a material of the headwear member 110. The retention loops 140 may be secured to the headwear member 110 directly, such as by stitching, or via loop attachments 142. The loop attachments 142 may be secured to the headwear member 110 such as by stitching. The loop attachments 142 may be constructed of the same or different fabric material as the headwear member 110. The design and location of the retention loops 140 may be configured to receive and help retain straps, wires, lines, or tubes of or connected to medical devices such as nasal adapters (prongs or masks) 176 of CPAP machines. The retention loops 140 may also help direct straps, wires, lines, or tubes away from the face of an infant. For example, as shown in FIGS. 21 and 22, nasal adapter straps 178 secured to a nasal adapter 176 are passed through or secured to the retention loops 140, which helps direct and maintain the nasal adapter straps 178 away from the face of an infant.

The headwear member 110 may be constructed of any one or more soft fabric materials known in the art. The fabric material may be any natural or synthetic fabric such as cotton, elastane or spandex, microfiber, polyester, rayon, silk, viscose, or wool, or any combination thereof. The fabric material may be a composite of open-celled, elastomeric, non-latex foam and engineered fabrics. For example, the composite may be Fabrifoam® (Fabrifoam, Exton, Pa.). The fabric may be woven, unwoven, or knit. A knit may be a smooth or ribbed knit. The material may be flexible, stretchable, migration resistant, hook receptive (i.e., micro-hooks from hook-and-loop fastener materials will attach to the material), wicking, breathable, cooling, fire retardant, machine washable, or any combination thereof.

In the construction and use of the headwear member 110, a material that is stretchable may help secure the headwear member 110 to an infant's head. A material that is stretchable may also provide versatile adjustability in both circumference of the headwear member 110 and height of the headwear member 110. A stretchable material may evenly apply an elastic-like grip around the circumference of the headwear member 110 for a comfortable fit that is also resistant to unintentional movement, such as rotating around an infant's head or slipping up or down on an infant's head.

Stretchable materials may include stretchable fabrics such as, for example, elastane or spandex, nylon, and ribbed knits. A ribbed knit may be highly stretchable across its series of ribs. If a ribbed knit is used in the construction of the headwear member 110 of FIGS. 1-4 such that the ribs are oriented approximately vertically (i.e., from the apex 128 to the lower portion 116 of the headwear member 110), the stretching ability is concentrated around the circumference of the headwear member 110. The fabric may stretch across the ribs by a factor of about two, about three, or about four. The fabric may stretch along the ribs to a lesser degree than across the ribs. For example, the fabric may stretch along the ribs by a factor of about one half to about one.

The stretch of a fabric may be limited by structures such as seams. The stretch of a fabric may also be limited by increasing the number of layers of fabric or overlaying a stretchable fabric and a non-stretchable fabric. Alternately, the fabric may be reinforced with elastic strips or bands that grip the infant's head.

In the construction and use of the headwear member 110, a migration-resistant fabric may help the headwear member 110 remain in place on an infant's head and may help reduce or prevent the unintentional movement of the headwear member 110, such as rotating around an infant's head or slipping up or down on an infant's head. Migration-resistant fabrics cling to or grip the surface with which they are in contact. Migration-resistant fabrics may include, for example, spandex and Fabrifoam®.

In the construction and use of the headwear member 110, a hook-receptive fabric may reduce the number of fasteners needed to secure the headwear member 110 to an infant's head or to attach devices, such as securing devices 200, to the headwear member 110. Hooks, such as Velcro® hooks, can directly engage hook-receptive fabrics. Hook-receptive fabrics may include, for example, fleece, flannel, terrycloth, and Fabrifoam®.

In the construction and use of the headwear member 110, a non-insulating fabric may help prevent an infant's body temperature from rising or reduce an infant's body temperature as compared to an insulating fabric. Non-insulating fabrics include fabrics that are wicking, breathable, and/or cooling.

A wicking fabric draws moisture away from skin and may also transfer it to a next, more outer, layer. Drawing moisture, usually perspiration, away from the skin helps regulate body temperature. For example, drawing moisture away from the skin helps a person feel or stay warmer in cool or cold environments and helps a person feel or stay cooler in warm or hot environments. A wicking fabric may help an infant regulate body temperature. Wicking fabrics may include, for example, cotton, microfiber, polyester, silk, and wool. Wicking fabrics may also include performance-engineered synthetic fabrics such as Fabrifoam®, Capilene® (Patagonia, Ventura, Calif.), FlashDry™ (The North Face, San Leandro, Calif.) and DriClime® (Marmot, Rohnert Park, Calif.).

A breathable fabric allows air to reach the skin and allows water vapor to escape from the fabric. Allowing air to reach the skin and allowing water vapor, usually from perspiration, to escape from the fabric helps to reduce body temperature and/or prevent body temperature from rising. A breathable fabric may help an infant stay cooler. Breathable fabrics may include, for example, cotton, linen, and silk. Breathable fabrics may also include performance-engineered synthetic fabrics such as Fabrifoam®, Gore-Tex® (breathable and waterproof; W. L. Gore & Associates, Elkton, Md.), OmniTech® (breathable and waterproof; Colombia Sportswear Co., Portland, Oreg.) and PolarTec® (breathable and insulating; Marmot, Rohnert Park, Calif.).

A cooling fabric allows heat to pass away from the skin through the fabric and does not reflect heat back to the skin. Allowing heat to pass through the fabric helps to reduce body temperature and/or prevent body temperature from rising. A cooling fabric may help an infant stay cooler. Cooling fabrics may include, for example, cotton, linen, and rayon.

The headwear member 110 may be constructed of one or more layers of soft materials, such as one layer. Each layer may be constructed of one or more pieces joined together by any means known in the art including, but not limited to, stitches, glue, tape, bonding, or any combination thereof. The one or more pieces may meet at one or more seams 130. In the exemplary embodiment depicted in FIGS. 1-4, the front side 112 of the headwear member 110 is constructed of one piece of Fabrifoam® and the back side 114 is constructed of one piece of cotton.

The positioning member 102 is secured to the headwear member 110 within a covering fabric. The covering fabric may be the fabric of the headwear member 110 or may be a separate piece of fabric, such as a casing. In the exemplary embodiment depicted in FIGS. 1-4, a portion of each positioning member 102, such as the portion adjacent the front side 112 of the headwear member 110, is constructed of one or more pieces of Fabrifoam®. A portion of each positioning member 102, such as the portion adjacent the back side 114 of the headwear member 110, is constructed of one or more pieces of cotton.

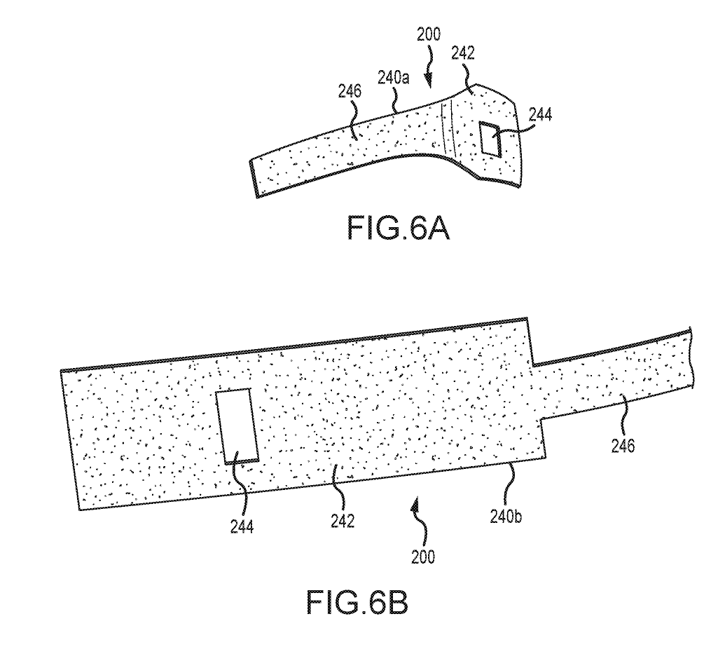

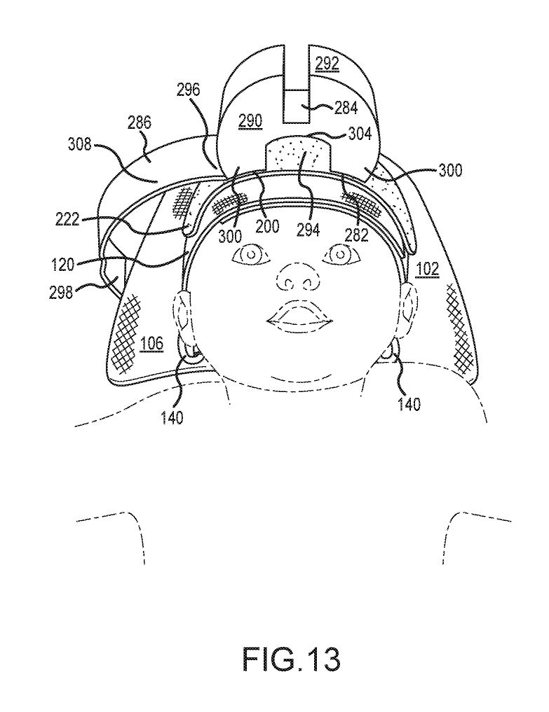

The positioning members 102 may be attached at lateral positions of the headwear member 110. In the exemplary embodiment depicted in FIGS. 1-4, a positioning member 102 is positioned on each of the left and right sides of the headwear member 110. The positioning members 102 meet or nearly meet at or near the apex 128. With reference to, for example, FIGS. 7 and 14, the positioning members 102 are positioned behind the ears of an infant when the head positioning aid 100 is in use.

The positioning members 102 minimize movement of an infant's head, maintain an infant's head in midline, and deflect an infant's moving head back to midline. The positioning members 102 may be positioned laterally when an infant's head is in a midline position, which diminishes pressure to the side of the face and head and thereby helps to prevent the development of dolichocephaly. The positioning members 102 may be positioned toward the front and back of the head when an infant is lying on its side, which diminishes pressure to the side of the head between the positioning members 102 and thereby helps to prevent the development of dolichocephaly. The positioning members 102 also help reduce, prevent, or treat IVH.

Each positioning member 102 is secured to the headwear member 110 at one or more junctures 126. A juncture 126 may be formed by any known means including, but not limited to, stitches, glue, tape, bonding, or any combination thereof. A juncture 126 may include a seam.

A juncture 126 may extend partially or completely along the length of the positioning member 102. A juncture 126 may extend to, near, or across the apex 128 of the headwear member 110. A juncture 126 may extend to or near the lower portion 116 of the headwear member 110. A juncture 126 may extend partially or completely along the width of the positioning member 102 at any point along the length of the positioning member 102.

Multiple junctures 126 may meet or overlap. One juncture 126 may transition into another, adjacent juncture 126. For example, if two or more junctures 126 are created by sewing, stitches may continue unbroken from one juncture 126 to an adjacent juncture 126.

A juncture 126 may secure a portion of the headwear member 110 to a minor portion, such as an edge, thin strip, small piece, or casing, of a positioning member 102. A juncture 126 may secure one portion of the fabric of the headwear member 110 to another portion of the fabric of the headwear member 110 such that a positioning member 102 is captured by the fabric of the headwear member 110, as in pocket. The pocket may be nominally larger than the positioning member 102 such that the positioning member 102 has no or limited room to move within the pocket.

The straps 120 of the headwear member 110 may be opened to apply or remove the head positioning aid 100. The straps 120 may be closed to help secure the head positioning aid 100 to the head of an infant. The straps 120 may be closed to help provide a surface to which medical devices can be attached.

When the straps 120 are in the open position (see FIG. 1), the straps 120 may be folded back such that they lay on top of a portion of the headwear member 110. The straps 120 may be wrapped underneath the positioning members 102.

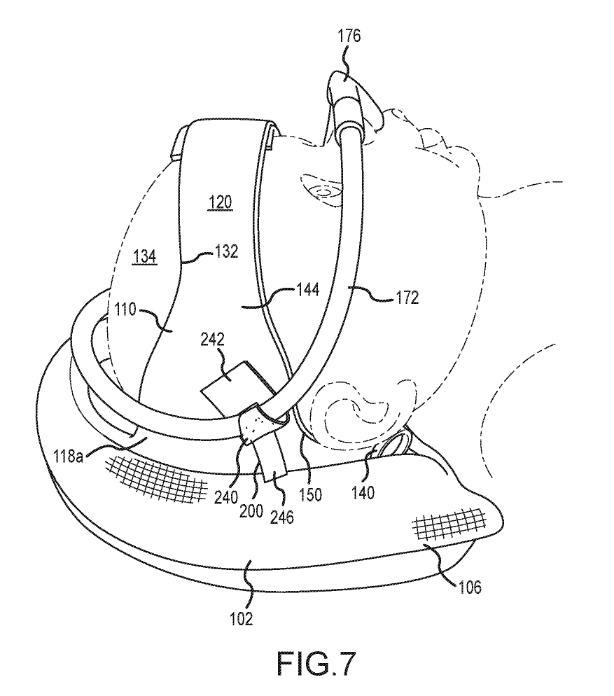

When the straps 120 are in the closed position (see, for example, FIGS. 7 and 8), the straps 120 lay substantially flat on the infant's skull. In some embodiments, the combined length of the straps 120 is at least long enough to traverse the distance between lateral flaps 118a, 118b across the forehead of an infant when the head positioning aid 100 is in use and the straps 120 are in the closed position. In some embodiments, the combined length of the straps 120 is at least long enough to traverse the distance between positioning members 102 across the forehead of an infant when the head positioning aid 100 is in use and the straps 120 are in the closed position. In the exemplary embodiment of FIG. 8, each strap 120 is slightly longer than half the distance from the lateral flap 118 to the center of an infant's forehead when the head positioning aid 100 is in use and the straps 120 are in the closed positon. The width of each strap 120 is wide enough to receive a securing device 200 (described below). The width of each strap 120 is not so wide as to cover the crown of an infant's head when the head positioning aid 100 is in use and the straps 120 are in the closed positon.

The straps 120 may be releasably secured to each other, to the headwear member 110, or to a positioning member 102 at one or more strap attachments 124. The strap attachments 124 may include, for example, buttons, snaps, hook-and-loop fasteners, or hook-and-eye fasteners. In the exemplary embodiment of FIG. 1, the strap attachment 124 is constructed of hook fasteners and is positioned on the bottom surface 146 at or near the free end 122 of a strap 120. In some embodiments, a second strap attachment 124, such as one constructed of loop fasteners, is positioned on the top surface 144 of the opposing strap 120. In some embodiments, a second strap attachment 124 is positioned on the lateral flap 118 or on a positioning member 102.

With reference again to the exemplary embodiment depicted in FIG. 1, the top surface 144 of the straps 120 is constructed of a hook-receptive material. A hook strap attachment 124 on the bottom surface 146 of one strap 120 can be secured to the hook-receptive top surface 144 of the opposing strap 120 by pressing the hook strap attachment 124 against the opposing strap 120. In some embodiments, the top surface of the lateral flap 118 is constructed of a hook-receptive material. A hook strap attachment 124 on a strap 120 can engage the hook-receptive surface of the lateral flap 118. In some embodiments, a portion of each positioning member 102, such as the portion adjacent the front side 112 of the headwear member 110, is constructed of a hook-receptive material. A hook strap attachment 124 on a strap 120 can engage the hook-receptive surface of the positioning member 102.

Securing the straps 120 helps to secure the head positioning aid 100 to an infant's head. Securing the straps 120 may help the headwear member 110 remain in place on an infant's head and may help reduce or prevent the unintentional movement of the headwear member 110, such as rotation around or slippage up or down on an infant's head. Securing the straps 120 may help provide a surface on which one or more securing devices 200 can be placed. In the construction and use of the straps 120, their shape, material, and position help protect the skin of an infant's face from abrasion or other damage caused by a securing device 200 or a medical device, such as a CPAP tube 174, touching or laying on the face.

With reference to FIGS. 7 and 14, when the straps 120 are in the closed position, the upper edge 132 of the straps 120 define a portion of an upper opening 134 in the headwear member 110. The lateral flap 118 and apex 128 define another portion of the upper opening 134. The upper opening 134 may be substantially circular or oval in shape. When the head positioning aid 100 is in use, the upper opening 134 is positioned over the top of the infant's head above the forehead. The upper opening 134 provides access to the scalp, which allows for placement, removal, and monitoring of scalp IVs. The upper opening 134 also permits temperature monitoring, regulation, and stabilization.

In some implementations, the head positioning aid 100 includes one or more securing devices 200. Each securing device 200 may help to hold a medical device or straps, wires, lines, or tubes of or connected to a medical device in a desired location on or near an infant's head. Each securing device 200 may help minimize or prevent movement of the devices or accessories. In the construction and use of a head positioning aid 100 in combination with a securing device 200, the head and neck of the infant are held in the desired position while medical devices or accessories are held in the desired position relative to the infant. Each of the head positioning aid 100 and securing device 200 may be configured for rapid set up and removal, which permits a medical provider to quickly position an infant as well as to quickly provide or withdraw medical treatments.

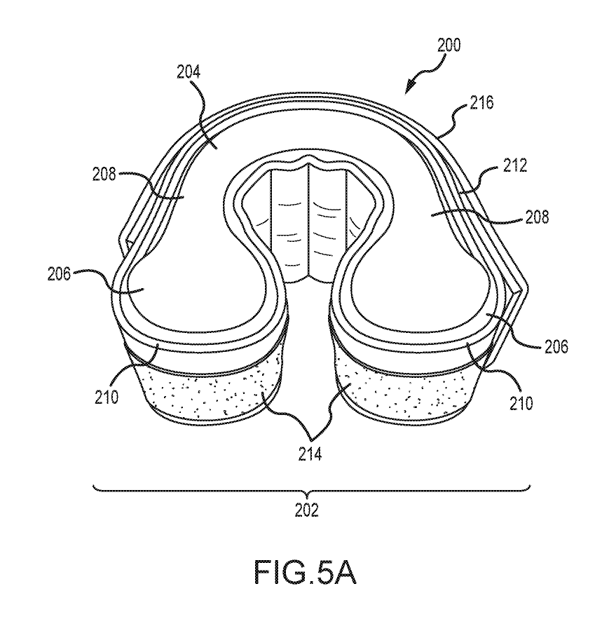

In one embodiment, as depicted in FIGS. 5A and 5B, the securing device 200 is a clamp securing device 202. The clamp 202 is substantially U-shaped with two legs 208 that each terminate in a foot 206. The clamp 202 may have a uniform depth, such as 0.25 inch to 2.5 inches. The clamp 202 may have a height of 0.5 inch to 3 inches and may have a width of 0.5 inch to 3 inches.

The clamp 202 includes a core 204, which may be constructed of a sturdy and resilient material that returns to its original shape, or close to its original shape, after being compressed or stretched. For example, the core 204 may be constructed of closed cell foam.

The core 204 may be of uniform width or, as in FIGS. 5A and 5B, the feet 206 may be wider than the legs 208. The width of the core may be 0.1 to 1.5 inches. The distance between the inside of the legs 208 may be enough to accommodate one or more straps, wires, lines, or tubes of or connected to a medical device, such as a CPAP tube 174. In one embodiment, the distance between the inside of the legs 208 may be 0.25 inch to 3 inches.

The clamp 202 includes an optional first clamp cover 210 that partially or completely surrounds the core 204. The first clamp cover 210 may be constructed of the same or different fabric material as the headwear member 110. The first clamp cover 210 is secured to the core such as by gluing or stitching. The first clamp cover 210 may help provide a smooth surface over the core 204 and may help increase the durability of the core 204.

The clamp 202 may include an optional second clamp cover 212 that partially or completely surrounds the first clamp cover 210 if a first clamp cover 210 is provided. The second clamp cover 212 may be constructed of the same or different material as the first clamp cover 210. In one embodiment, the outer surface 216 of the second clamp cover 212 is constructed of a hook-receptive material, which permits a portion of a medical device to be secured to the second clamp cover 212 with hook fasteners.

The clamp 202 may include one or more clamp attachments 214 positioned on one or both feet 206. In the embodiment depicted in FIGS. 5A and 5B, the clamp attachments 214 are hook fasteners and are secured to the first clamp cover 210 by an adhesive. In other embodiments, the clamp attachments 214 are secured, such as by stitching or gluing, to the core 204 or the second clamp cover 212. Each clamp attachment 214 may be the same size and shape or a different size and size. A clamp attachment 214 may be as wide as or narrower than the depth of a foot 206. A clamp attachment 214 may be any length. In the embodiment of FIGS. 5A and 5B, each clamp attachment 214 may be longer than the width of a foot 206 such that covers a portion of both the inside and the outside of each leg 208.

In the construction and use of a clamp 202, the material and shape may permit the clamp 202 to be stretched around a strap, wire, line, or tube of or connected to a medical device, such as a CPAP tube 174. The clamp 202 may also help decrease movement of the strap, wire, line, or tube.

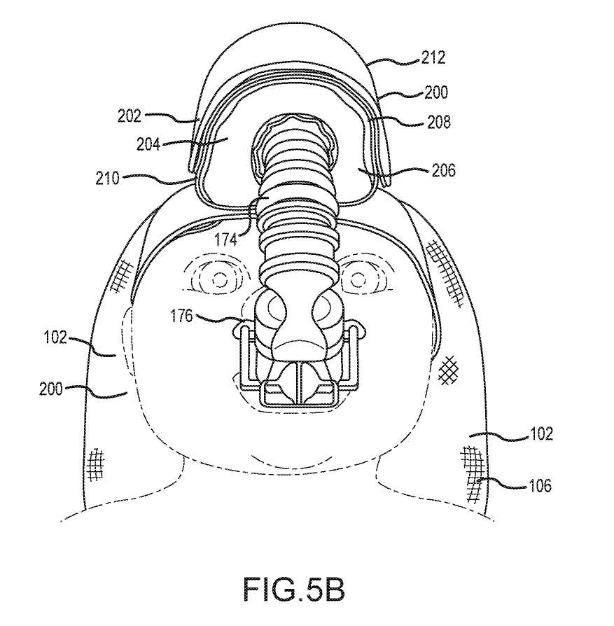

In one embodiment, the securing device 200 is a tie wrap securing device 240a, 240b, which may be constructed of hook fasteners on at least one surface such that it can engage a hook-receptive fabric. The tie wrap 240a, 240b includes a head portion 242, a tail portion 246, and an aperture 244. The head portion 242 may be any size and shape, the tail portion 246 may be any shape and is long enough to pass through the aperture 244, and the aperture 244 may be any size and shape that the tail portion 246 can pass through. In the embodiments shown in FIGS. 6-8, each of the head portions 242, tail portions 246, and apertures 244 may be substantially rectangular in shape and the entire length of the tie wraps 240a, 240b may be 1 inch to 6 inches.

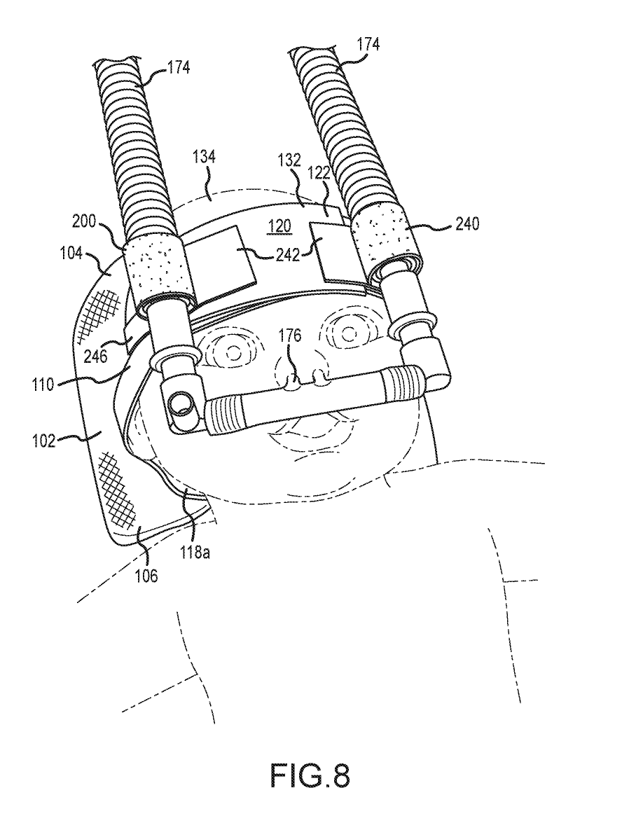

In one embodiment, as depicted in FIGS. 9A and 9B, the securing device 200 is a cradle securing device 252. The cradle 252 may include a cradle cover 256 around two lateral support rails 260 positioned on either side of a tube receiving slot 254. The cradle 252 may have a height of 0.5 inch to 3 inches, a width (as measured across both lateral support rails 260 and the tube receiving slot 254) of 1 inch to 3 inches, and a uniform depth, such as 0.25 inch to 2.5 inches.

The tube receiving slot 254 may extend the entire depth of the cradle 252. The width and the height of the tube receiving slot 254 are great enough to accommodate one or more straps, wires, lines, or tubes of or connected to a medical device, such as a CPAP tube 174. In one embodiment, the tube receiving slot 254 may have a width of 0.5 inch to 2.5 inches and a height of 0.5 inch to 3 inches.

Each lateral support rail 260 may be constructed of a sturdy material that may also be compressible or resilient, such as closed cell foam. Each lateral support rail 260 may be substantially cylindrical or cuboidal in shape. Each lateral support rail 260 may extend the entire depth of the cradle 252. Each lateral support rail 260 may have a width of 0.25 inch to 2 inches and a height of 0.5 inch to 3 inches.

The cradle cover 256 may be constructed of the same or different fabric material as the headwear member 110. A portion of the cradle cover 256 may be secured to the lateral support rails 260 such as by gluing or stitching. In the embodiment depicted in FIGS. 9A and 9B, a fixed end 266 of the cradle cover 256 may be secured to the interior 270 of a lateral support rail 260 between the lateral support rail 260 and the tube receiving slot 254. The cradle cover 256 may wrap around the top 274 and exterior 272 of the lateral support rail 260 to the bottom 264 of the cradle 252, may traverse the bottom 264 of the cradle 252 from one lateral support rail 260 to the tube receiving slot 254 and to the second lateral support rail 260, may wrap around the exterior 272 and top 274 of the second lateral support rail 260 to the top 262 of the cradle 252.

In one embodiment, as shown in FIG. 9A, a cradle fastener 258, such as a Velcro® fastener, may be secured to an inner surface 278 of the cradle cover 256 at or near a free end 268 of the cradle cover 256. In another embodiment, the cradle cover 256 is constructed of two-layer Velcro® such that the outer surface 276 of the cradle cover 256 can engage the inner surface 278 of the cradle cover 256.

When the cradle 252 is in the open position (see FIG. 9A), the free end 268 may lay back on a portion of the cradle cover 256. When the cradle 252 is in the closed position (see FIG. 9B), the free end 268 may traverse the tube receiving slot 254 from one lateral support rail 260 to the other lateral support rail 260 such that a lid 280 is formed on the cradle 252. The free end 268 may be secured to the cradle cover 256 by engaging the cradle fastener 258 and the hook-receptive outer surface 276 of the cradle cover 256 on the top 274 of a lateral support rail 260. The free end 268 may be secured to the cradle cover 256 by engaging opposing fasteners on the inner surface 278 of the free end 268 and the of the cradle cover 256 on the top 274 of a lateral support rail 260. Straps, wires, lines, or tubes may be captured in the tube receiving slot under the lid 280 of the cradle cover 256.

The cradle 252 may include one or more cradle attachments (not shown) positioned on the bottom 264 of the cradle 252. The cradle attachment may be a hook fastener and may be secured to the cradle cover 256, such as by gluing or sewing. A cradle attachment may be as wide as or narrower than the depth of the cradle 252. A cradle attachment may be as long as or shorter than the width of the cradle 252 (as measured across both lateral support rails 260 and the tube receiving slot 254). The cradle attachment may help secure the cradle 252 to the headwear member 110, such as to a strap 120.

In the construction and use of a cradle 252, the material and design may help maintain straps, wires, lines, or tubes of or connected to a medical device, such as CPAP tubes 174, in a desired location and decrease movement of the tubes 174 away from the desired location.

The cradle 252 may be used in conjunction with a headwear member attachment strip 222. The headwear member attachment strip 222 may be constructed of hook fasteners on at least a bottom surface 224 such that it can engage a hook-receptive fabric of the headwear member 110. The top surface 232 may be constructed of hook fasteners, loop fasteners, or a fabric material. The top 232 and bottom surfaces 224 may together be constructed of two-ply Velcro®. The headwear member attachment strip 222 may be substantially rectangular in shape with a length greater than its width. The headwear member attachment strip 222 may have a width less than the width of a strap 120 and a length less than the combined lengths of the straps 120. For example, the headwear member attachment strip 222 may be 0.5 inch to 4 inches in width and 1 to 10 inches in length. The headwear member attachment strip 222 may have the same construction and dimensions as described above for use with the clamp 202.

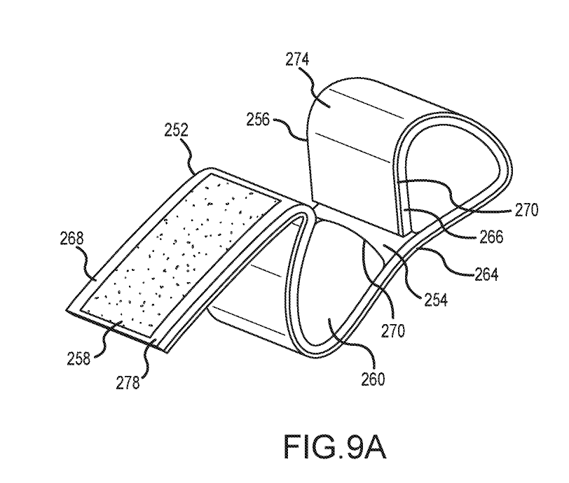

In one embodiment, as depicted in FIGS. 10-14, the securing device 200 is a bridge securing device 282. The bridge 282 may include a body 290 and a bridge securing strip 286. The bridge 282 may have an overall semicylindrical shape when the bridge securing strip 286 is in the closed position. The bridge 282 may have a uniform depth, such as 0.25 inch to 2.5 inches, a height of 0.5 inch to 3 inches, and a width of 1 inch to 3 inches. The body 290 may be constructed of a sturdy material that may also be compressible or resilient, such as closed cell foam.

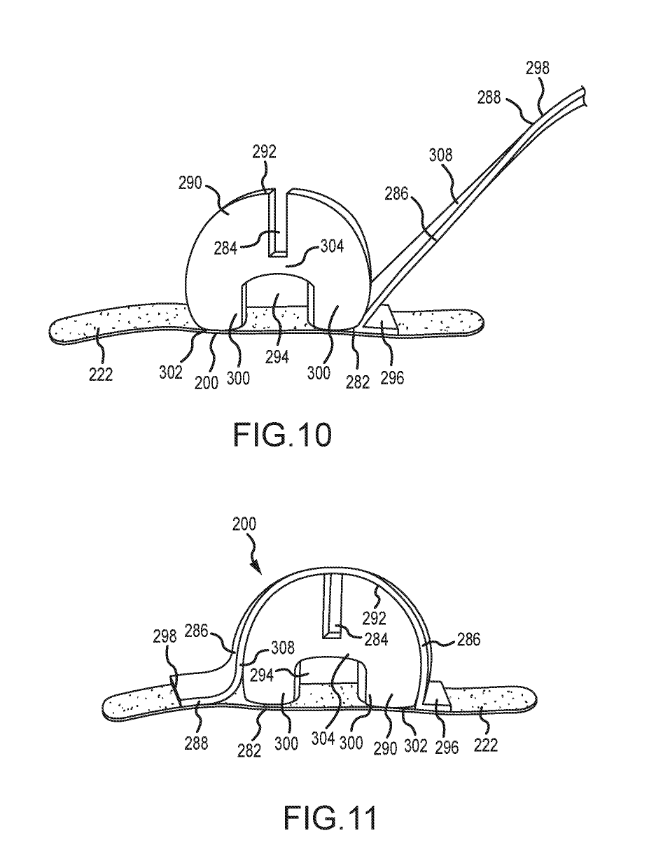

The body 290 may include a tube receiving slot 284 and a recess 294 positioned between two feet 300. The width and the height of the tube receiving slot 254 are great enough to accommodate one or more tubes, lines, wires, or straps of or connected to a medical device, such as a CPAP tube 174. The tube receiving slot 254 may extend for a portion of the height of the body 290 from the top wall 304 of the recess 294 to a top edge 292 of the body 290 creating an opening or gap in the top edge 292 as shown in FIG. 12. For example, the tube receiving slot 284 may extend for 20-75% of the height of the body 290. In one embodiment, the tube receiving slot 254 may have a height of 0.1 inch to 2 inches and a width of 0.1 inch to 2 inches.

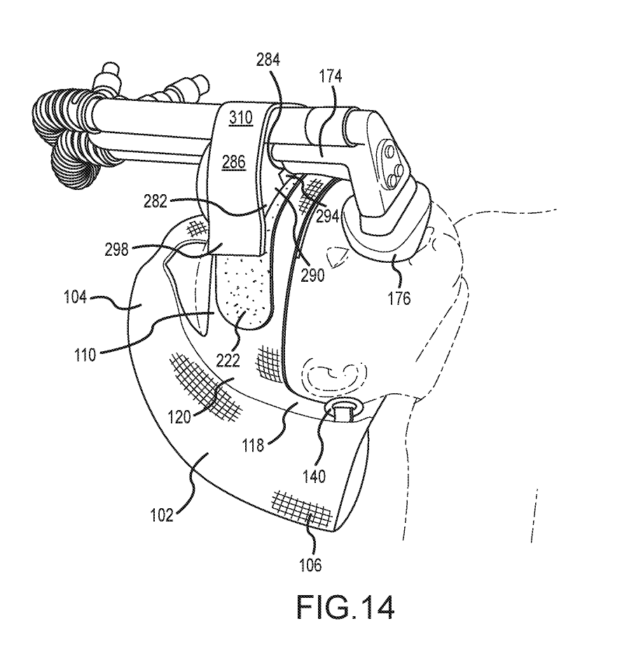

In some embodiments, as shown in FIG. 15, the tube receiving slot 284 is wider at the portion at the top wall 304 than at the top edge 292, such as in a tear-drop shape. A slit 306 may extend laterally away from tube receiving slot 284 in one or both directions. Each slit 306 may be substantially parallel to the plane of the top wall 304 or may follow the arc of the top edge 292 of the body 290. In the design and construction of the body 290, the slit 306 may help absorb weight or pressure from a captured medical device, such as a CPAP tube 174, and thereby prevent the pressure from being applied to an infant's head, which can cause bruising or deformation.

The top wall 304 may traverse the top of the recess 294 between two feet 300 positioned on each lateral side of the recess 294 and beneath the tube receiving slot 284. The top wall 304 provides a web of material between the recess 294 and the tube receiving slot 284. The top wall 304 may help to distribute weight or force of the tubes or other devices placed in the tube receiving slot 284 laterally to the feet 300 when tubes, lines, wires, or straps of or connected to a medical device are received in the tube receiving slot 284.

The recess 294 may be any shape such as rectangular as shown in FIG. 12. In the design and construction of the recess 294, the width and height are great enough to permit the body 290 to receive and contain at least some of the tubes, lines, wires, or straps as shown in FIG. 14. The top wall 304 can also be compressed into the recess 294 to adjust the desired height of the tubes or other devices above the infant's head. For example, as shown in FIG. 14, the body 290 may be compressed toward the headwear member 110 when the headwear member 110 and bridge securing device 282 are in use on the head of an infant and tubes, lines, wires, or straps of or connected to a medical device are received in the tube receiving slot 284. Compressing the body 290 may partially or fully collapse the recess 294 below the top wall 304 and may result in a decrease in the height of the body 290. The recess 294 may be collapsed more or less to help position tubes at various angles in relation to the head of an infant. In one embodiment, the recess 294 may have an uncompressed height of 0.1 inch to 2 inches and a width of 0.1 inch to 2 inches.

The body 290 of bridge securing device 282 may include one or more foot attachments 302 positioned on the bottom of the feet 300. The foot attachment 302 may be a hook fastener and may be secured to the feet 300, such as by gluing or sewing. A foot attachment 302 may be as wide as or narrower than the width of a foot 300. A foot attachment 302 may be as long as or shorter than the depth of a foot 300. In one embodiment, the foot attachment 302 may help secure the bridge 282 to the headwear member 110, such as to a strap 120. In another embodiment, as shown in FIGS. 10, 11, 13 and 14, the foot attachment 302 may help secure the bridge 282 to a headwear member attachment strip 222. The headwear member attachment strip 222 may have the same construction and dimensions as described above for use with the clamp 202. The depth of the bridge 282 may be the same as or different from the width of the headwear member attachment strip 222.

The bridge securing strip 286 may be constructed of the same or different fabric material as the headwear member 110. In the embodiment depicted in FIGS. 10, 11, 13 and 14, a fixed end 296 of the bridge securing strip 286 may be secured to a headwear member attachment strip 222 such as by gluing or stitching. In one embodiment, as shown in FIGS. 10, 11, 13 and 14, a bridge fastener 288, such as a Velcro® fastener, may be secured to an inner surface 308 of the bridge securing strip 286 at or near a free end 298 of the bridge securing strip 286.

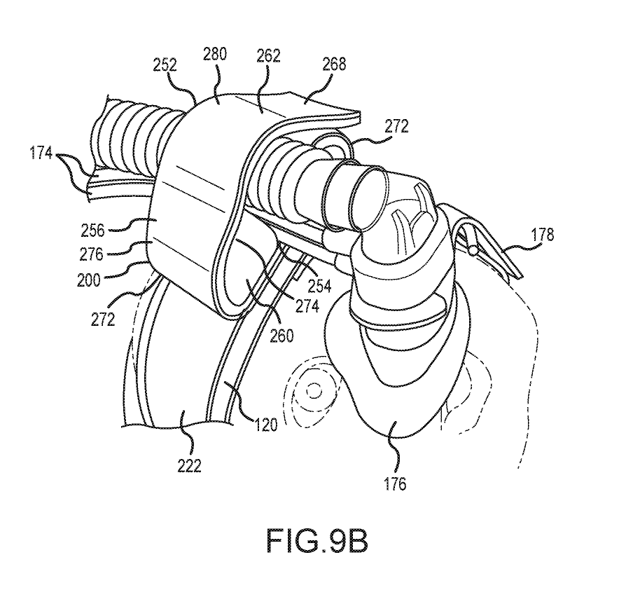

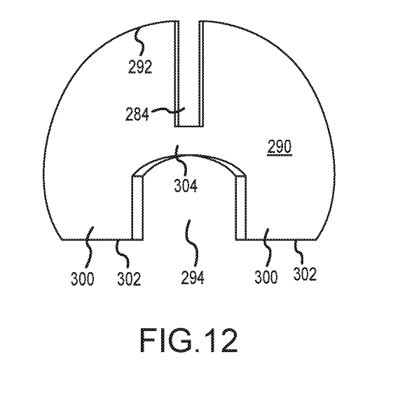

When the bridge securing device 282 is in the open position (see FIGS. 10 and 13), the free end 298 of the bridge securing strip 286 may lay back on a portion of the headwear member 110. When the bridge 282 is in the closed position (see FIGS. 11 and 14), the free end 298 may traverse the tube receiving slot 284 across the top edge 292 of the body 290 such that a closure 310 is formed over the tube receiving slot 284 in the body 290. In one embodiment, the free end 298 may be secured to the headwear member 110 by engaging the bridge fastener 288 to the hook-receptive outer surface 276 of the headwear member 110. In another embodiment, as shown in FIG. 14, the free end 298 may be secured to the headwear member attachment strip 222 by engaging the bridge fastener 288 and the hook-receptive top surface 232 of the strip 222. Tubes, lines, wires, or straps may be captured in the tube receiving slot 284 under the closure 310 of the securing strip 286.

In the construction and use of a bridge securing device 282, the material and design may help maintain tubes, lines, wires, or straps of or connected to a medical device, such as CPAP tubes 174, in a desired location and decrease movement of the tubes 174 away from the desired location. The material and design may also help enable the bridge securing device 282 to accommodate, for example, CPAP tubes 174 that are connected at various heights and angles to nasal adapters 176 such as a mask or prongs. In some embodiments, the material and design may also help distribute the weight or force of tubes received in the tube receiving slot 284 throughout the body 290 and thus across a larger surface area of the infant's head, thus minimizing the risk of bruising or deformation due to such pressure.

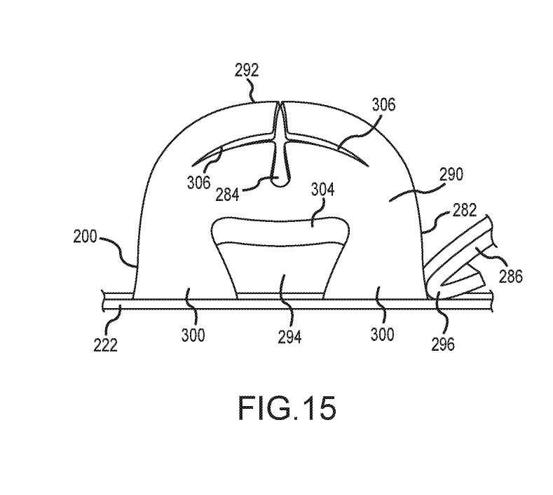

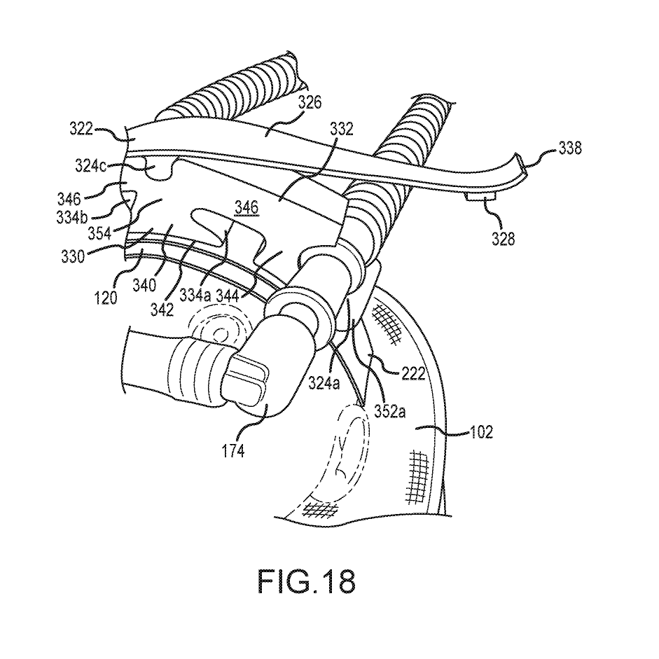

In one embodiment, as depicted in FIGS. 16-19, the securing device 200 is a goalpost toploader securing device 322. The goalpost toploader 322 may include a body 330 and a goalpost toploader securing strip 326. The goalpost toploader 322 may have an overall arc or crescent shape when the goalpost toploader securing strip 326 is in the closed position (see FIG. 19). The body 330 may have a uniform depth, such as 0.25 inch to 2.5 inches, a width of 2 inches to 6 inches, and a height of 0.25 inch to 2.25 inches. The body 330 may be constructed of a sturdy material that may also be bendable, compressible, or resilient, such as closed cell foam.

The body 330 may include at least two tube receiving slots 324. In some embodiments, a tube receiving slot 324a, 324b is positioned near each lateral end 352a, 352b of the body 330. In some embodiments, a tube receiving slot 324c is positioned at or near the center of the width of the body 330. The shape and dimensions of the tube receiving slots 324 are such that the slots 324 can accommodate one or more tubes, lines, wires, or straps of or connected to a medical device, such as a CPAP tube 174. The shape and dimension of each tube receiving slot 324 may be the same or different. The tube receiving slots 324 may be defined within a portion of the height of the body 330 from a top edge 332 of the body 330 toward the bottom surface 342 creating openings or gaps in the top edge 332 bordered by lateral walls 344 as shown in FIG. 16. For example, the tube receiving slot 324 may extend for 20-95% of the height of the body 330. In one embodiment, the tube receiving slots 324a, 324b may have a height of 0.2 inch to 2 inches and a width of 0.2 inch to 1.75 inches. In one embodiment, the tube receiving slot 324c may have a height of 0.1 inch to 1 inch and a width of 0.1 inch to 1 inch.

The body 330 may include at least one aperture 334 positioned at or near the bottom surface 342 of the body 330 between two tube receiving slots 324. In some embodiments, as shown in FIG. 16, the body 330 includes an aperture 334a, 334b positioned on either side of a foot 340 and between two tube receiving slots 324a, 324c; 324b, 324c. Each aperture 334a, 334b may be positioned such that is shares a lateral wall 344 with a tube receiving slot 324. A top wall 346 may traverse each aperture 334a, 334b and extend to or near one or more lateral walls 344.

In some embodiments, the foot 340 may be positioned beneath a tube receiving slot 324c. A top portion 354 may traverse the top of the foot 340 between two apertures 334a, 334b and beneath the tube receiving slot 324c. The foot 340 may be any shape such as trapezoidal as shown in FIG. 16. The top portion 354 provides a web of material between the tube receiving slot 324c and the foot 340. The top portion 354 may help to distribute weight or force of the tubes or other devices placed in the tube receiving slot 324c laterally across the foot 340 or laterally to the apertures 334a, 334b when tubes, lines, wires, or straps of or connected to a medical device are received in the tube receiving slot 324c.

Each aperture 334a, 334b may be any shape such as trapezoidal as shown in FIG. 16. The apertures 334a, 334b may have the same shape or different shapes. In the design and construction of the apertures 334a, 334b, the width and height are great enough to permit the body 330 to receive and contain a member of the tubes, lines, wires, or straps as shown in FIG. 19. The top wall 346 can also be compressed into each aperture 334a, 334b to adjust the desired height of the tubes or other devices above the infant's head. For example, as shown in FIG. 19, the body 330 may be compressed toward the headwear member 110 when the headwear member 110 and goalpost toploader securing strip 326 are in use on the head of an infant and tubes, lines, wires, or straps of or connected to a medical device are received in the tube receiving slots 324. Compressing the body 330 may partially or fully collapse each aperture 334a, 334b below the top wall 304 and may result in a decrease in the height of the body 330. Each aperture 334a, 334b may be collapsed more or less to help position tubes at various angles in relation to the head of an infant. In one embodiment, each aperture 334a, 334b may have an uncompressed height of 0.1 inch to 2 inches.

As shown in FIGS. 16-19, the goalpost toploader securing device 322 may be used in conjunction with a headwear member attachment strip 222. The headwear member attachment strip 222 may have the same construction and dimensions as described above for use with the clamp 202. The depth (thickness) of the goalpoast toploader 322 may be the same as or different from the width of the headwear member attachment strip 222.

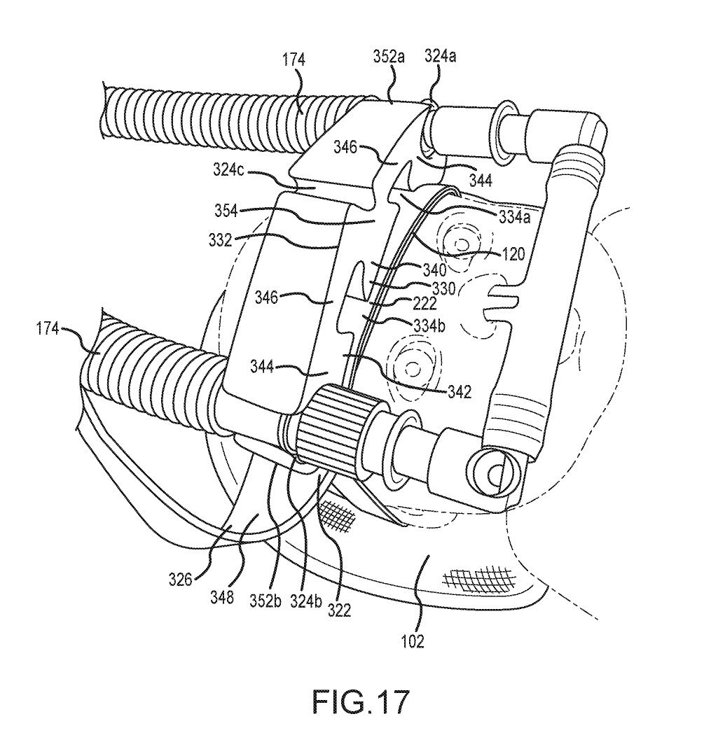

The goalpost toploader securing strip 326 may be constructed of the same or different fabric material as the headwear member 110. In the embodiment depicted in FIG. 16, a fixed end 336 of the goalpost toploader securing strip 326 may be secured to a headwear member attachment strip 222 such as by gluing or stitching. In one embodiment, as shown in FIG. 18, a goalpost fastener 328, such as a hook-and-loop fastener, may be secured to an inner surface 348 of the goalpost toploader securing strip 326 at or near a free end 338 of the goalpost toploader securing strip 326.

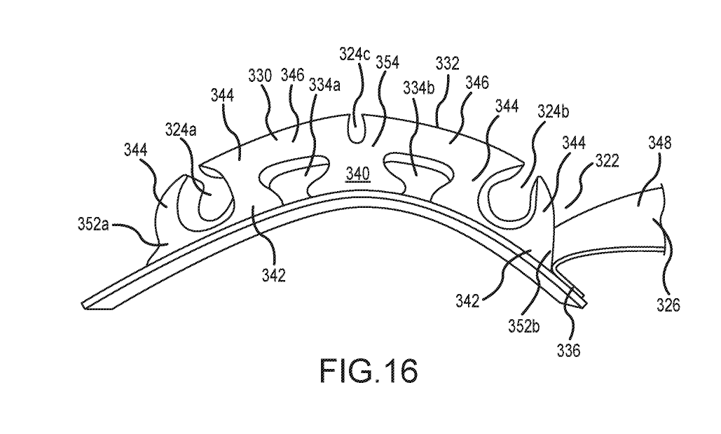

When the goalpost toploader securing device 322 is in the open position (see FIG. 17), the free end 338 of the goalpost toploader securing strip 326 may lay back on a portion of the headwear member 110. When the goalpost toploader 322 is in the closed position (see FIG. 19), the free end 338 may traverse the tube receiving slots 324 across the top edge 332 of the body 330 such that a closure 350 is formed over the tube receiving slots 324a, 324b in the body 330. In one embodiment, the free end 338 may be secured to the headwear member 110 by engaging the goalpost fastener 328 to the hook-receptive outer surface 276 of the headwear member 110. In another embodiment the free end 338 may be secured to the headwear member attachment strip 222 by engaging the goalpost fastener 328 and the hook-receptive top surface 232 of the strip 222. Tubes, lines, wires, or straps may be captured in the tube receiving slots 324a, 324b, 354 under the closure 350 of the securing strip 326.

In the construction and use of a goalpost toploader securing device 322, the material and design may help maintain tubes, lines, wires, or straps of or connected to a medical device, such as CPAP tubes 174, in a desired location and decrease movement of the tubes 174 away from the desired location. The material and design may also help enable the goalpost toploader securing device 322 to accommodate, for example, CPAP tubes 174 that are connected at various heights and angles to nasal adapters 176 such as a mask or prongs. In some embodiments, the material and design may also help distribute the weight or force of tubes received in the tube receiving slots 324a, 324b throughout the body 330 and thus across a larger surface area of the infant's head, thus minimizing the risk of deformation due to such pressure. In some embodiments, the construction and design of the goalpost toploader securing device 322 may permit quick attachment or detachment of CPAP tubes 174 and may enable a medical provider to rapidly respond to the changing medical status of an infant, such as by rapidly starting or removing CPAP.

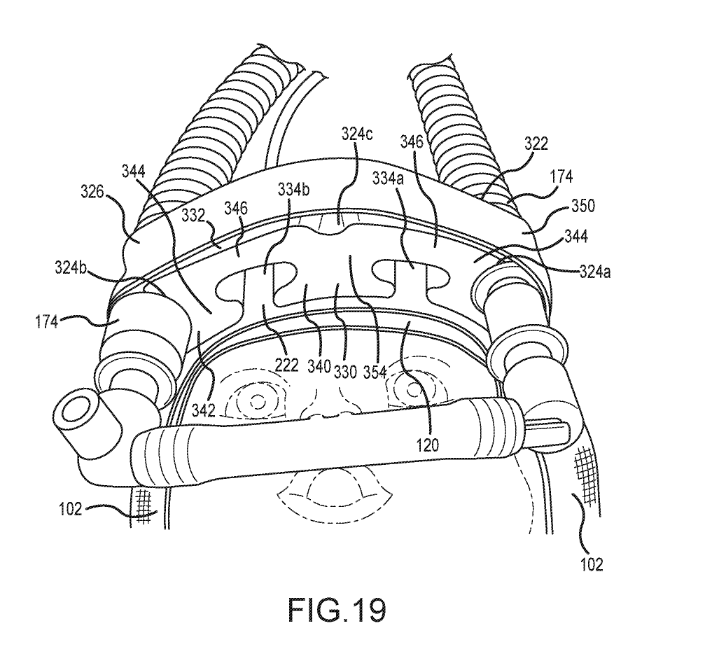

In some embodiments, as shown in FIGS. 20-22, at least one nasal adapter strap 178 is provided, which may help secure a nasal adapter 176 to the head positioning aid 100 without irritating or damaging the skin of an infant's face. The nasal adapter straps 178 may be constructed of the same or different material as the headwear member 110. At least the surface of the nasal adapter straps 178 that faces the infant's skin is a soft non-abrasive material such as Fabrifoam®. The nasal adapter straps may be generally rectangular-shaped strips with a width of 0.1 to 1 inch and a length of 2 to 10 inches.

In some embodiments, and with continued reference to FIGS. 20-22, the nasal adapter strap 178 includes a nasal end 186 and a distal end 188. The nasal end 186 may be configured to be secured to a nasal adapter 176. A nasal loop 180 is positioned along the length of the nasal adapter strap 178 between the nasal end 186 and distal end 188. The nasal loop 180 may be constructed of a stiff material such as plastic. The nasal adapter strap 178 includes at least two slots 194 positioned at or near the distal end 188 and the nasal end 186 is secured by passing the end 186 through the slots 194. When the strap 178 is secured, it folds back on itself such that the top surfaces 196 are in contact with each other and the nasal end 186 may rest on or near the positioning member 102. The length of the portion of the strap 178 that traverses a side of the face, such as the cheek, of an infant may be adjusted by pulling the nasal end 186 more or less through the slots 194. In the construction and use of the embodiment depicted in FIGS. 20-22, the nasal loop 180 can be positioned anywhere along the length of the strap 178 and the strap 178 is fully adjustable to accommodate any size infant.

The distal end 188 of each nasal adapter strap 178 is configured to pass through or be secured to a retention loop 140. In some embodiments, the distal end 188 is secured with a hook 190 as shown in FIGS. 20-22. The hook 190 may be constructed of a stiff material such as plastic or metal. In the construction and use of a nasal adapter strap 178 that includes a hook 190, the strap 178 can be quickly attached to and released from a headwear member 110. The quick attachment and release helps enable a medical provider to rapidly respond to the changing medical status of an infant, such as by rapidly starting or removing CPAP. In some embodiments, as shown in FIG. 14, the distal end 188 is secured by passing it through the retention loop 140 and fastening the distal end 188 to the same nasal adapter strap 178, such as by stitching, tying, knotting, or engaging a fastener. In some embodiments, the distal end 188 passes through a retention loop 140 and is secured at another location, such as on the strap 120.

In the construction and use the nasal adapter strap 178, the strap 178 is adjustable to fit all sizes of infants' heads. The strap 178 is also configured to attach to any and all types or brands of medical device interfaces, such as CPAP interfaces.

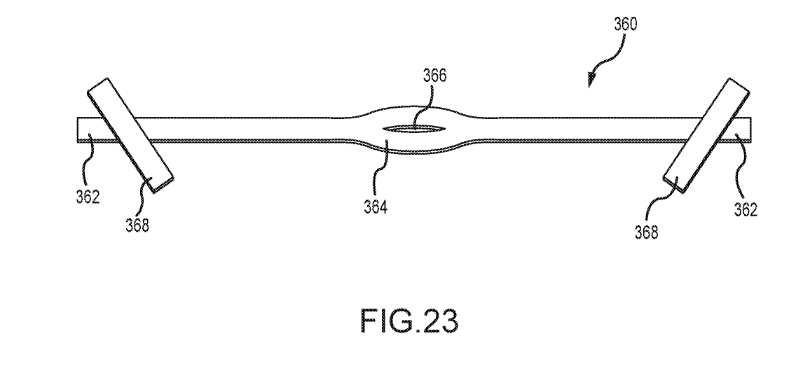

In some implementations, as shown in FIG. 23, the head positioning aid 100 includes a chin strap 360. The chin strap 360 may help secure the head positioning aid 100 to the infant's head and may help balance or counter forces exerted on the head positioning aid 100 or the infant's head by an attached medical device. The chip strap 360 may also held hold an infant's mouth closed when air is being delivered to the infant's nose via a medical device such as a nasal cannula or CPAP machine. In the embodiment depicted in FIG. 23, the chin strap 360 is a substantially rectangular strip with distal ends 362 as well as a wider center portion 364 positionable over the chin. The center portion 364 may include a split or notch 366, which may help the chin strap 360 conform to the angle of the chin. A chin strap securing strip 368 may be separate from or fixed to the chin strap 360, such as by sewing. The chin strap securing strip 368 may be constructed of hooks, such as of Velcro®, and may be able to engage the hook-receptive surface of the headwear member 110. When the chin strap 360 is in use with the headwear member 110 and the chin strap securing strip 368 is fixed to the chin strap 360, the strip 368 may be positioned along the length of the strap 360 such that the strip 368 is able to engage the headwear member 110 but not contact the face of the user. For example, the chin strap securing strips 368 may be fixed at or near the distal ends 362 of the chin strap 360. The chin strap 360 is secured to the headwear member 110, such as by capturing the strap 360 at or near a distal end 362 between the headwear member 110 and the chin strap securing strip 368. Securing the chin strap 360 to the headwear member 110 helps secure the head positioning aid 100 to the infant's head and helps keep the infant's mouth closed.

In some implementations, the head positioning aid 100 includes a strap flap (not shown). The strap flap may help protect the skin of an infant's face from abrasion or other damage caused by a securing device 200 or a medical device, such as a CPAP tube 174, touching or laying on the face. The strap flap may be any size and shape that helps protect the skin, such a substantially rectangular or semicircular.

The strap flap may be constructed of the same fabric material as the headwear member 110, or may be constructed of a different fabric material. A strap flap may be positioned on a strap 120, such as at or near the upper edge 132 and may extend into the upper opening 134 when the strap flap is in use. A strap flap may be positioned at or near the lower edge of a strap 120 and extend toward or over the cheek. A strap flap may be permanently or releasably secured to a strap 120, such as by stitches, glue, tape, bonding, fasteners, or any combination thereof.

In the construction and use of a strap flap, its size, shape, material, and position help protect the skin of an infant's face from abrasion and other damage caused by a securing device 200 or a medical device, such as a CPAP tube 174, touching or laying on the face or scalp.

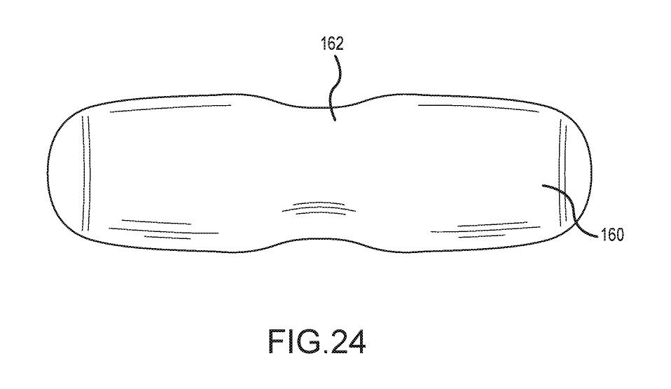

In some implementations, the head positioning aid 100 includes a support member 160. With reference to FIG. 24 the support member 160 may be substantially cylindrical in shape. The support member 160 may have a length greater than its width (diameter). The support member 160 may be the same length as, shorter than, or longer than the distance between the lower portions 104 of the positioning members 102. Thus, the support member 160 may extend beyond the lower portion 104 of one or more positioning members 102, extend to the lower portion 104 of one or more positioning members 102, or terminate short of the lower portion 104 of one or more positioning members 102.

A support member 160 may have the same circumference along the entire length of the support member 160, or the circumference may vary along the length of the support member 160. A support member 160 may include a depression 162 at or near the midpoint of its length such that the circumference of the support member 160 is decreased at the depression 162. The depression 162 may be wide enough to accommodate an infant's neck. The depression 162 may be on one or more sides of the support member 160. For example, as show in FIG. 24, the depression 162 may be on all sides of the support member 160 such that it uniformly decreases the circumference of the support member 160.

The support member 160 may be comprised of a filler. The filler may be, for example, solid foam, memory foam, stuffing, batting, down, synthetic down-like material, gel, or a combination thereof. The filler may be resilient such that it returns to its original shape, or close to its original shape, after being compressed. The filler may be of sufficient quantity, compactness, or firmness that it resists complete compression by the weight of an infant's head or neck. The filler may be partially compressible, but has enough compression resistance that it helps the support member 160 support an infant's head or neck. The compression resistance is effective at any weight of an infant's head.

The support member 160 may include a casing, such as a fabric casing. The casing may completely or partially surround the filler. The casing may be constructed with one more seams.

A support member 160 may be physically separate from but used in conjunction with the head positioning aid 100. Alternatively, a support member 160 may be permanently or releasably secured to the head positioning aid 100. The support member 160 may be secured to the lower portion 116 of the back side 114 of headwear member 110, such as between the positioning members 102. A headwear member 110 may include a pocket or sleeve into which the support member 160 is placed or removed to releasably secure the support member 160. The support member 160 may be secured to the headwear member 110 by stitches, glue, tape, bonding, fasteners, or any combination thereof. Fasteners may include, for example, buttons, snaps, hook-and-loop fasteners, or hook-and-eye fasteners. By way of example, but not limitation, Velcro® may be attached to part or all of the length of a support member 160, and Velcro® may be attached to part of or the entire hem 148 of the back lower portion 136 of the headwear member 110. The support member 160 is thus releasably secured to the headwear member 110 by engaging opposing Velcro® pieces.

In the construction and use of a support member 160, its size, shape, compressibility, and attachment to the headwear member 110 help support an infant's head or neck, and help promote an open airway and uniform distribution of respiratory support to the lungs.

Methods of Use of the Head Positioning Aid

By way of example, but not limitation, the head positioning aid 100 of FIGS. 1-4 may be used to support an infant in a midline supine position according to the following procedure. An infant is placed on its back and its head is positioned on a head positioning aid 100 having the straps 120 in the open position, or the head positioning aid 100 is guided underneath the infant's head. Placing an infant on the head positioning aid 100 or sliding the head positioning aid 100 down the back of the infant's head minimizes disturbance to and stress on the infant.

The head positioning aid 100 may be positioned with the back side 114 of the headwear member 110 facing the incubator mattress or other surface. The apex 128 of the headwear member 110 may be positioned behind the crown of the head. The back lower portion 136 of the headwear member 110 may be positioned behind the back of the neck.

The lateral flap 118 may be drawn up and in toward the center of the face such that the lateral flap 118 may be positioned next to or on top of the infant's ears. The free end 122 of a strap 120 may be drawn across the infant's forehead toward the opposing positioning member 102 and may be laid substantially flat against the infant's forehead. The bottom surface 146 of the strap 120 contacts the infant's skin without irritating or damaging it. When a second strap 120 is present, the free end 122 of the second strap 120 may be drawn toward the opposing positioning member 102 and may be laid substantially flat against the infant's forehead or on top of the top surface 144 of the previously positioned strap 120. The second strap 120 may be secured to the first strap 120 by pressing a strap attachment 124 positioned on the bottom surface 146 of the second strap 120 against the hook-receiving top surface 144 of the first strap 120 to engage the two surfaces 144, 146.

When the straps 120 are engaged, the upper edges 132 define a portion of an upper opening 134. The upper opening 134 permits access to the scalp for placement, removal, or monitoring of scalp IVs and for regulating temperature.

The positioning members 102 may be positioned on either side of the infant's head, behind the ears. The positioning members 102 maintain an infant's head in midline and deflect an infant's moving head back to midline. The positioning members 102 follow the curvature of the infant's head such that the lower portions 116 may fall behind or in front of the tops of the shoulders. This arrangement of the lower portions 116 helps provide additional lateral support to the positioning members 102 for maintaining the head in a midline position. When the infant's head rests on or rolls onto a positioning member 102, the positioning member 102 deflects the infant's head back to midline. The shape, thickness, and/or firmness of the positioning members 102 may help to deflect an infant's head back to midline. Maintaining an infant's head in midline helps to promote optimal cerebral blood flow and uniform distribution of respiratory support to the lungs. The positioning members 102 are positioned laterally, which diminishes pressure to the side of the face and head and thereby helps to prevent the development of dolichocephaly.

The head positioning aid 100 of FIGS. 1-4 may be used to support an infant in a midline position while the infant is lying on its side according to the following procedure. The head positioning aid 100 may be positioned and secured to the infant's head as described above for FIGS. 1-4 except that access to the lateral flap 118 positioned on the side of the head on which the infant is lying may be limited and the flap 118 is therefore not drawn toward the center of the face.

The positioning members 102 may be positioned toward the front and back of the infant's head. The infant's head may lie on one or more positioning members 102, or may lie between the positioning members 102. The positioning members 102 support and maintain an infant's head in a midline position while the infant is lying on its side. Supporting and maintaining an infant's head in a midline position helps to reduce pressure to the side of the head between the positioning members 102 and thereby helps to prevent the development of dolichocephaly. The side of the head on which the infant is positioned may be alternated, which also helps to prevent the development of dolichocephaly.

The head positioning aid 100 of FIGS. 1-4 may include one or more securing devices 200. By way of example, but not limitation, a head positioning aid 100 may be used with a clamp securing device 202 as shown in FIG. 5B to support an infant's head in midline, as well as to secure straps, wires, lines, or tubes of or connected to medical devices and to guide them away from an infant's face, according to the following procedure.

The head positioning aid 100 may be applied to an infant's head according to any of the methods described above. A clamp 202 may be positioned over previously placed straps, wires, lines, or tubes of or connected to a medical device, such as CPAP tubes 174, by grasping the outer surface 216 of the second clamp cover 212 and pressing down toward the head positioning aid 100 such that the feet 206 are displaced outward around the tubes 174. The feet 206 return to or close to their original positon when the tubes 174 are seated within the clamp 202 such that the tubes 174 may be positioned between the legs 208 and the feet 206 are positioned partially between the tubes 174 and the headwear member 110. The clamp 202 may be secured to the headwear member 110 by pressing down to engage the clamp attachments 214 on the feet 206 with the hook-receptive front side 112 of the headwear member 110. The tubes 174 are thereby captured by the clamp 202 and the legs 208 help to prevent lateral movement of the tubes 174. The clamp may be positioned on a strap 120 approximately in the center of the infant's forehead in line with its nose and in line with a previously placed nasal adapter 176.

In some implementations, a clamp 202 may be used in conjunction with a headwear member attachment strip 222 to help increase the strength and rigidity of the connection between the clamp 202 and the headwear member 110. The headwear member attachment strip 222 may be applied to the headwear member 110 approximately in the center of the forehead on the top surface 144 of a closed strap 120. A clamp 202 may be then applied over tubes or wires as described above except that the clamp attachment 214 may engage the hook-receptive top surface 232 of the headwear member attachment strip 222 instead of engaging the strap 120.

A previously placed strap, wire, line, or tube of or connected to a medical device, such as a nasal cannula tube 172, may be guided through the passage 235 formed by the capture loop 236. The tube 172 may be captured by pressing a releasable end 248 of the securing strip 238 against the headwear member attachment strip 222 to engage the complimentary hook and loop fasteners on the bottom surface 237 of the securing strop 238 and the top surface 232 of the headwear member attachment strip 222.

In another alternative method, a nasal cannula tube 172, or similar device, may be captured between the bottom surface 224 of the headwear member attachment strip 222 and the headwear member 110, thereby securing the tube 172 to the head positioning aid 100.

As another example, the head positioning aid 100 of FIGS. 1-4 may be used with a tie wrap securing device 240b as shown in FIGS. 6-8 to support an infant's head in midline, as well as to secure straps, wires, lines, or tubes of or connected to medical devices and to guide them away from an infant's face, according to the following procedure.

The head positioning aid 100 may be applied to an infant's head according to any of the methods described above. A tie wrap 240b may be positioned under a previously placed strap, wire, line, or tube such that the hook fastener surface faces the head positioning aid 100. The tail portion 246 may be drawn around the, for example, nasal cannula tube 172, passed through the aperture 244, drawn away from the head portion 242, and pulled to tighten the tie wrap 240b around the tube 172. The head portion 242 and exposed portion of the tail portion 246 may be pressed against the hook-receptive front side 112 of the headwear member 110 or positioning member 102 at any desired location.

As another example, the head positioning aid 100 of FIGS. 1-4 may be used with a cradle securing device 252 as shown in FIG. 9B to support an infant's head in midline, as well as to secure straps, wires, lines, or tubes of or connected to medical devices and to guide them away from an infant's face, according to the following procedure.

The head positioning aid 100 may be applied to an infant's head according to any of the methods described above. A cradle 252 may be secured to a closed strap 120 approximately in the center of the infant's forehead. A CPAP tube 174 or similar device may be guided into and through the tube receiving slot 254. The CPAP tube 174 may be captured by drawing the free end 268 of the cradle cover 256 around the tubes 174 to the distal lateral support rail 260. A cradle fastener 258 may be pressed against the top 274 of the rail 260 to engage complimentary hook and loop fasteners.

The cradle 252 may be used in conjunction with a headwear member attachment strip 222. The headwear member attachment strip 222 may be applied to a closed strap 120 as described above. The cradle 252 may be positioned as described above except that the bottom 264 of the cradle 252 may be secured to the headwear member attachment strip 222 instead of to the closed strap 120.

As another example, the head positioning aid 100 of FIGS. 1-4 may be used with a bridge securing device 282 as shown in FIGS. 13 and 14 to support an infant's head in midline, as well as to secure tubes, lines, wires, or straps of or connected to medical devices and to guide them away from an infant's face, according to the following procedure.

The head positioning aid 100 may be applied to an infant's head according to any of the methods described above. A bridge 282 may be secured to a closed strap 120 approximately in the center of the infant's forehead by engaging the foot attachments 302 with the strap 120. One or more CPAP tubes 174 or a similar device may be guided into the tube receiving slot 284. The CPAP tube 174 may be captured by drawing the free end 298 of the bridge securing strip 286 over the tubes 174 toward the distal foot 300. A bridge fastener 288 may be pressed against the strap 120 to engage complimentary hook and loop fasteners.

The bridge 282 may be used in conjunction with a headwear member attachment strip 222 as shown in FIGS. 13 and 14. The headwear member attachment strip 222 is applied to a closed strap 120 as described above. The bridge 282 is positioned as described above except that the foot attachments 302 are secured to the headwear member attachment strip 222 instead of to the closed strap 120.

As another example, the head positioning aid 100 of FIGS. 1-4 may be used with a goalpost toploader securing device 322 as shown in FIGS. 17-19 to support an infant's head in midline, as well as to secure tubes, lines, wires, or straps of or connected to medical devices and to guide them away from an infant's face, according to the following procedure.

The head positioning aid 100 may be applied to an infant's head according to any of the methods described above. A goalpost toploader 322 may be secured to a strap 120 in the closed position approximately in the center of the infant's forehead. One or more CPAP tubes 174 or a similar device may be guided into one or more tube receiving slots 324. The CPAP tube 174 may be captured by drawing the free end 338 of the goalpost toploader securing strip 326 over the tubes 174 toward the distal lateral end 352 of the body 330. A goalpost fastener 328 may be pressed against the strap 120 to engage complimentary hook and loop fasteners.

The goalpost toploader 322 may be used in conjunction with a headwear member attachment strip 222 as shown in FIGS. 17-19. The headwear member attachment strip 222 may be applied to a strap 120 in the closed position as described above. The goalpost toploader 322 may be positioned as described above except that the bottom surface 342 of the body 330 may be secured to the headwear member attachment strip 222 instead of to the strap 120 in the closed position.

As another example, the head positioning aid 100 of FIGS. 1-4 may be used with a nasal adapter strap 178 to support an infant's head in midline, as well as to secure a nasal adapter 176 of a medical device, such as of a CPAP machine. In one embodiment, as shown in FIGS. 21 & 22, the nasal adapter strap 178 may be used to secure a nasal adapter 176 and limit or prevent movement of the nasal adapter 176 according to the following procedure.

The head positioning aid 100 may be applied to an infant's head according to any of the methods described above. The nasal loop 180 may be hooked over a hook on the nasal adapter 176. The distal end 188 of the nasal adapter strap 178 may be threaded through one or both slots 194 and pulled taught. The slots 194 help secure the strap 178 in place but also permit quick and easy adjustment of the tension on the strap 178. The preceding steps may be repeated for a nasal adapter strap 178 positioned on the other side of the infant's head. Securing two straps 178 helps to hold the nasal adapter 176 in the desired location over the infant's nose and minimizes or prevents unintentional movement of the nasal adapter 176.

The head positioning aid 100 of FIGS. 1 and 2 may include a strap flap. By way of example, but not limitation, a head positioning aid 100 may be used with a strap flap to support an infant's head in midline, as well as to protect the skin of the face of an infant, according to the following procedure.

The head positioning aid 100 may be applied to an infant's head according to any of the methods described above. When a device that extends beyond the bounds of the straps 120, such as a securing device 200 or medical device, is used with the head positioning aid 100, the strap flap may be positioned between the device and the infant's skin. The strap flap helps protect an infant's skin from abrasion or other damage caused by a securing device 200 or a medical device.

The head positioning aid 100 of FIGS. 3 and 4 may also be used to support an infant in a midline position according to the following alternative procedure. An infant is placed on its back or on its side on the back side 114 of a head positioning aid 100 (see FIG. 4), or the head positioning aid 100 is guided underneath an infant's head. The head positioning aid 100 may be positioned with its front side 112 facing the incubator mattress or other surface and with its back side 114 facing the infant's head. The apex 128 of the headwear member 110 may be positioned behind the crown of the head. The lower portion 116 of the headwear member 110 may be positioned behind the back of the neck. The positioning members 102 may be positioned laterally when an infant's head is in a midline supine position. The positioning members 102 may be positioned toward the front and back of one side of the head when an infant's head is in midline and the infant is lying on its side. The positioning members 102 maintain the infant's head in midline as described above. The straps 120 may or may not be utilized in this alternative procedure and may be positioned in the open position (see FIG. 3) or tucked under the front side 112 of the headwear member (see FIG. 4).

The head positioning aid 100 of FIGS. 1-4 may include a support member 160 as depicted in FIG. 24. By way of example, but not limitation, a head positioning aid 100 may be used with a support member 160 to support an infant's head in a midline position, as well as to support the neck of an infant and promote an open airway, according to the following procedure.

The head positioning aid 100 may be applied to or behind an infant's head according to any of the methods described above. The support member 160 may already be attached to the headwear member 110, or it may be slid behind the neck of an infant after the head positioning aid 100 has been positioned. The neck of the infant may rest on the depression 162 in the support member 160. The support member 160 helps support an infant's head or neck. The support member 160 also helps promote an open airway and uniform distribution of respiratory support to the lungs, such as when an infant is on a ventilator.

Head Positioning Aid Kits

By way of example, but not limitation, the head positioning aid 100 of FIGS. 1-4 may be provided as part of a kit. A kit can include a head positioning aid 100 and one or more securing devices 200. The securing devices 200 may be one or more clamp 202, tie wrap 240a, 240b, cradle 252, or bridge 282 securing devices in any combination. In some embodiments, a kit may also include one or more of a nasal adapter strap 178, support member 160, or chin strap 360. A launderable bag may also be provided in which the kit components can be transported, stored, or washed. In some embodiments, a kit includes instructions for using the head positioning aid 100, securing devices 200, support member 160, chin strap 360, or launderable bag.

The article “a” or “an” preceding a term, as used herein, refers to one or more of that term. As such, the terms “a” or “an”, “one or more”, and “at least one” should be considered interchangeable herein.

All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present invention, and do not create limitations, particularly as to the position, orientation, or use of the invention. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. The exemplary drawings are for purposes of illustration only and the dimensions, positions, order and relative sizes reflected in the drawings attached hereto may vary.

The above specification, examples and data provide a complete description of the structure and use of exemplary embodiments of the invention as defined in the claims. Although various embodiments of the claimed invention have been described above with a certain degree of particularity, or with reference to one or more individual embodiments, those skilled in the art could make numerous alterations to the disclosed embodiments without departing from the spirit or scope of the claimed invention. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

What is claimed is:

1. A head positioning aid comprising
a headwear member further comprising
at least one strap attached to a lateral portion of the headwear member;
two longitudinally elongated, compression-resistant, positioning members spaced laterally apart and attached adjacent to opposite edges of the lateral portion of the headwear member; and
at least one securing device for securing a medical device to the head of an infant,
wherein the at least one strap is configured to secure the head positioning aid to a head of an infant by traversing the forehead of the infant, the strap is configured to receive medical devices while maintaining access to the infant's skull and, when secured, the strap defines an opening between an upper edge of the strap, the lateral portion, and an apex of the headwear member,
the lateral portion of the headwear member extends along a side portion of the head when the headwear member is in use, and
the positioning members are positioned adjacent the head and are configured to support the head of the infant in a midline position with respect to the spine of the infant when the infant is supine or lying on its side.

2. The head positioning aid of claim 1, wherein the headwear member is constructed of a migration-resistant material.

3. The head positioning aid of claim 1 or claim 2, wherein the headwear member is constructed of a hook-receptive material.

4. The head positioning aid of claim 3, wherein the material is Fabrifoam®.

5. The head positioning aid of claim 3, wherein the strap comprises a hook fastener configured to engage the hook-receptive material.

6. The head positioning aid of any one of claims 1-5, wherein the securing device is releasably attached to the head positioning aid.

7. The head positioning aid of any one of claims 1-6, wherein the securing device is configured to receive at least one tube for providing continuous positive airway pressure to an infant.

8. The head positioning aid of any one of claims 1 to 7, wherein the securing device is a clamp comprising a substantially U-shaped core surrounded by a cover to which fasteners are secured.

9. The head positioning aid of claim 8, wherein the fasteners are hook fasteners configured to secure the clamp to the head positioning aid.

10. The head positioning aid of any one of claims 1 to 7, wherein the securing device is a cradle comprising
two lateral support rails defining a slot therebetween wherein the slot is configured to receive medical tubes;
a top strap; and
fasteners secured to the top strap and configured to secure the cradle to the head positioning aid.

11. The head positioning aid of claim 10, wherein the top strap has a fixed end and a free end and the tubes are captured in the slot by fastening the fixed end of the top strap over the slot and to the support rails.

12. The head positioning aid of any one of claims 1 to 7, wherein the securing device is a bridge comprising
a body having a top edge, a bottom edge, lateral portions, and a central wall oriented substantially parallel to the bottom edge;
a covering strap; and
fasteners secured to the bottom edge and configured to secure the bridge to the head positioning aid, wherein
the top edge, lateral portions, and central wall define a slot therebetween, and
the slot is configured to receive medical tubes.

13. The head positioning aid of claim 12, wherein the bottom edge, lateral portions, and central wall define a recess therebetween and the lateral wall is collapsible into the recess when tubes are received in the slot.

14. The head positioning aid of any one of claims 1 to 7, wherein the securing device is a goalpost toploader comprising
a top edge;
a bottom portion;
a first exterior wall and a second exterior wall, each exterior wall extending between the top edge and the bottom portion; and
at least one interior wall extending between the top edge and the bottom portion, wherein
the top edge, bottom portion, first exterior wall, and an interior wall define a first aperture,
the top edge, bottom portion, second exterior wall, and an interior wall define a second aperture, and
the apertures are configured to receive medical tubes.

15. The head positioning aid of claim 14, wherein the goalpost toploader comprises
a first interior wall and a second interior wall;
the top edge, bottom portion, first exterior wall, and first interior wall further define the first aperture;
the top edge, bottom portion, second exterior wall, and second interior wall further define the second aperture; and
the top edge, bottom portion, first interior wall, and second interior wall define a third aperture, wherein
the first and second apertures are each configured to receive a CPAP tube, and
the third aperture is configured to receive an additional medical tube.

16. The head positioning aid of any one of claims 1 to 7, wherein the securing device is a nasal adapter strap comprising
a first end configured to be secured to the head positioning aid;
a second end; and
a loop positioned between the first end and the second end and configured for attachment to a nasal prong or a mask of the medical device.

17. The head positioning aid of claim 16, wherein the headwear member further comprises a pair of laterally positioned retention loops configured for passing the second end of the nasal adapter strap therethrough.

18. A kit comprising
the head positioning aid of claim 1; and
at least one of a nasal adapter strap, support member configured to support the neck of an infant when the head positioning aid is in use, chin strap, or launderable bag.

19. A method of securing a medical device to the head of an infant comprising
placing a head positioning aid under the head of an infant lying supine or on its side, wherein the head positioning aid comprises
two longitudinally elongated, compression-resistant, positioning members spaced laterally apart from each other; and
two straps that extend laterally between the positioning members;
engaging a fastener on one strap with the second strap to form a closed strap configuration across the forehead of the infant and to secure the head positioning aid to the infant's head;
releasably attaching a securing device to the straps in the closed configuration, wherein the securing device comprises
an upper portion;
a lower portion; and
at least two lateral portions positioned between the upper and lower portions, wherein
the upper portion, the lower portion, and the lateral portions define an opening therebetween configured to receive a medical device; and
securing a medical device to the headwear member by placing a first portion of the medical device in the opening in the securing device.

20. The method of claim 19, comprising
securing a second portion of the medical device to the head of an infant using a nasal adapter strap, the nasal adapter strap further comprising
a first end configured to be secured to the head positioning aid; and
a second end configured to be secured to the second portion of the medical device,
releasably attaching the first end of the nasal adapter strap to the head positioning aid at a location near the ears of the infant; and
relesasably attaching the second end of the nasal adapter strap to the second portion of the medical device positioned near the nose of the infant.
